(19) United States
(12) Patent Application Publication
(10) Pub. No.: US 2016/0321422 A1
(43) Pub. Date: Nov. 3, 2016

(54) SYSTEM AND METHOD OF MANAGING PORTABLE LASER THERAPY APPARATUS

(71) Applicant: K-LASER, LLC, Franklin, TN (US)

(72) Inventor: Richard Albright, Franklin, TN (US)

(21) Appl. No.: 15/208,751

(22) Filed: Jul. 13, 2016

Related U.S. Application Data
(63) Continuation-in-part of application No. 14/798,106, filed on Jul. 13, 2015.
Application No. 14/798,106 is a continuation-in-part of application No. 13/470,976, filed on May 14, 2012, now abandoned.
Application No. 13/470,976 is a continuation-in-part of application No. 14/798,121, filed on Jul. 13, 2015.
Application No. 14/798,121 is a continuation-in-part of application No. 13/470,976, filed on May 14, 2012, now abandoned.
(60) Provisional application No. 62/191,903, filed on Jul. 13, 2015.

(51) Int. Cl. G06F 19/00; A61B 18/22; A61N 5/06

(52) U.S. Cl. CPC G06F 19/3412; A61N 5/06; A61B 18/22; A61N 2005/063; A61N 2005/067

(57) ABSTRACT

Provided is a method of using a computer system to manage a remote laser emitting apparatus used in medical treatments, including the computer system receiving, through computer data communication, apparatus identification information associated with a remotely located laser emitting apparatus, and user identification information associated with a user of the laser emitting apparatus, the computer system storing the apparatus identification information and user identification information, the computer system correlating, in response to receiving data regarding a purchase of one or more treatment credits by the user, the purchased treatment credits to the laser emitting apparatus associated with the user, and the computer system communicating the purchased treatment credits to the laser emitting apparatus through computer data communication, wherein the purchased treatment credits authorize the laser emitting apparatus to perform a number of treatments corresponding to the purchased treatment credits.

### CROSS-REFERENCE TO RELATED APPLICATIONS

This application claims the benefit of U.S. Provisional Patent Application Ser. No. 62/191,903, filed on Jul. 13, 2015, and is a continuation-in-part of and claims priority to U.S. patent application Ser. No. 14/798,106, filed Jul. 13, 2015, and U.S. patent application Ser. No. 14/798,121, filed on Jul. 13, 2015, both of which are continuation-in-part applications of and claim priority to U.S. patent application Ser. No. 13/470,976, filed May 14, 2012. The entire content of all the foregoing applications is incorporated by reference herein.

### FIELD OF INVENTION

The present general inventive concept relates generally to a portable apparatus to perform laser therapy, and, more particularly, a portable apparatus to apply lasers of different wavelengths and powers to living tissue for therapeutic treatment and/or surgery.

### BACKGROUND

Laser light therapy has become increasingly popular in physiotherapy and surgery applications due to the many benefits available through the application of laser light. Laser light can be used to treat a variety of problems, ranging from relatively mild conditions, such as acne and skin wrinkling, to more complex problems lying deep under the skin, including afflictions of both organs and bones. In many cases, the application of laser therapy may negate the need for conventional pharmaceutical and/or surgical procedures. Different powers, wavelengths, and frequencies are used to target the distinct tissue types associated with the different medical conditions being treated. With the many physical benefits available over the large range of these powers, wavelengths, and frequencies, there exists a need for a device to deliver a large number of different combinations of these values in order to treat a wide variety of conditions. Further, in order for the device to be readily adapted in home and field use as well as medical office and clinical conditions, the device should be readily portable, updatable, and relatively easy to use.

### BRIEF SUMMARY

The present general inventive concept provides a readily portable laser emitting apparatus to conveniently apply laser emissions to a patient for therapeutic treatment and/or surgery. The portable laser emitting apparatus includes one or more laser sources, and may be controlled so as to apply a variety of levels of laser emissions according to different desired therapeutic and surgical procedures.

Additional aspects and advantages of the present general inventive concept will be set forth in part in the description which follows, and, in part, will be obvious from the description, or may be learned by practice of the present general inventive concept.

The foregoing and/or other aspects and advantages of the present general inventive concept may be achieved by a portable laser emitting apparatus to be used in physiotherapy and/or surgery, the apparatus including a readily portable housing, one or more laser sources of same or different wavelengths provided in the housing, and a flexible waveguide extending from the housing to transmit laser light from the one or more laser sources to a target area.

The flexible waveguide may include an optical fiber with a core size of approximately 200 um, and an NA of approximately 0.15 to 0.37.

The apparatus may further include a handpiece, provided at a distal end of the flexible waveguide, configured to emit the laser light to the target area.

The handpiece may be configured to be selectively controlled to deliver the laser light in a focus mode or a zoom mode.

The apparatus may further include at least two detachable members to be selectively attached to the handpiece according to selection of the focus mode or the zoom mode.

At least one of the detachable members may be a twist control that is twisted to adjust a contact area of the transmitted laser light in the zoom mode.

A spot size of the laser light delivered in the zoom mode may be adjustable from approximately 1 to 5 cm².

The detachable members may be mechanically or magnetically coupled to the handpiece.

The apparatus may further include a combiner to combine light from the one or more laser sources into the laser light transmitted by the flexible waveguide.

The flexible waveguide may be provided with a pliable metal sheath surrounding the flexible waveguide and control wiring connected to the handpiece.

The one or more laser sources may include a first laser source to transmit laser light having a wavelength of approximately 660 nm at a power up to approximately 100 mW, and a second laser source to transmit laser light having a wavelength of approximately 800 nm in a power range of approximately 0.1 to 12.0 W.

The one or more laser sources may further include a third laser source to transmit laser light having a wavelength of approximately 970 nm at a power range of approximately 0.1 to 12.0 W.

The second and/or third laser source may transmit at a power range of approximately 0.1 to 8.0 W.

The one or more laser sources may further include a fourth laser source to transmit laser light having a wavelength of approximately 905 nm at a power range of approximately 0.1 to 12.0 W.

The frequencies of the laser light may be adjustable between approximately 1 to 20,000 Hz in approximately 1 Hz increments.

Any of the one or more laser sources may be controlled to emit the laser light separately or concurrently.

The apparatus may further include a carrying handle provided to the housing so that the apparatus may be transported by hand by a user.

The apparatus may further include a data storage to store a plurality of predetermined settings of wavelength and power combinations to be emitted from the one or more laser sources, and a controller to control the one or more laser sources to operate according to the predetermined settings.

The apparatus may further include a communication terminal to receive data updates for the controller and/or data storage.

The communication terminal may be a USB port.

The communication terminal may perform wireless communication.

The apparatus may further include a touch screen user interface.

The apparatus may further include a rechargeable battery to supply power to the apparatus.

The one or more laser sources may be light emitting diodes.

The foregoing and/or other aspects and advantages of the present general inventive concept may also be achieved by a method of using a computer system to manage a remote laser emitting apparatus used in medical treatments, including the computer system receiving, through computer data communication, apparatus identification information associated with a remotely located laser emitting apparatus, and user identification information associated with a user of the laser emitting apparatus, the computer system storing the apparatus identification information and user identification information, the computer system correlating, in response to receiving data regarding a purchase of one or more treatment credits by the user, the purchased treatment credits to the laser emitting apparatus associated with the user, and the computer system communicating the purchased treatment credits to the laser emitting apparatus through computer data communication, wherein the purchased treatment credits authorize the laser emitting apparatus to perform a number of treatments corresponding to the purchased treatment credits.

The foregoing and/or other aspects and advantages of the present general inventive concept may also be achieved by a portable laser emitting apparatus to be used in physiotherapy and/or surgery, the apparatus including a readily portable housing, one or more laser sources of same or different wavelengths provided in the housing, a flexible waveguide extending from the housing and configured to transmit laser light from the one or more laser sources to a target area, a data storage configured to store a plurality of predetermined settings of wavelength and power combinations to be emitted from the one or more laser sources, data corresponding to past usage of the laser emitting apparatus, and treatment credit information regarding whether the laser emitting apparatus is authorized to perform one or more treatments, a communication terminal configured to receive data updates for the controller and/or data storage from a remote device management system, and a controller configured to control the one or more laser sources to operate according to the predetermined settings in response to the laser emitting apparatus being authorized to perform a treatment, the past usage data, and communication with the remote device management system to share the usage data and/or receive new treatment credits.

The foregoing and/or other aspects and advantages of the present general inventive concept may also be achieved by a system to perform laser therapy, the system including a portable laser emitting apparatus configured to perform laser therapy, and a remotely located computer system configured to receive, through computer data communication, apparatus identification information associated with the laser emitting apparatus, and user identification information associated with a user of the laser emitting apparatus, to store the apparatus identification information and user identification information, to correlate, in response to receiving data regarding a purchase of one or more treatment credits by the user, the purchased treatment credits to the laser emitting apparatus associated with the user, and to communicate the purchased treatment credits to the laser emitting apparatus through computer data communication, wherein the purchased treatment credits authorize the laser emitting apparatus to perform a number of treatments corresponding to the purchased treatment credits.

Other features and aspects may be apparent from the following detailed description, the drawings, and the claims.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 illustrates a portable laser emitting apparatus according to an example embodiment of the present general inventive concept;

FIG. 2 is a schematic illustration of some of the components of the portable laser emitting apparatus of FIG. 1, according to an example embodiment of the present general inventive concept;

FIG. 3 illustrates example elements and functions of the handpiece of the portable laser emitting apparatus according to an example of the present general inventive concept;

FIG. 4 is a graph illustrating the application of the laser light in the continuous wave mode;

FIG. 5 is a graph illustrating the application of the laser light in the frequency modulated, or pulsed emission, mode;

FIG. 6 is a graph illustrating the application of the laser light in the intense super pulse mode;

FIG. 7 illustrates an available treatments display used to track and control operations of the portable laser emitting apparatus according to an example embodiment of the present general inventive concept;

FIG. 8 illustrates a purchase options display used to allow a user to choose from a plurality of purchase options according to an example embodiment of the present general inventive concept;

FIG. 9 illustrates an expected revenue display to track and control operations of the portable laser emitting apparatus according to an example embodiment of the present general inventive concept;

FIG. 10 illustrates a history display used to track and control operations of the portable laser emitting apparatus according to an example embodiment of the present general inventive concept;

FIG. 11 illustrates number of treatments information that may be displayed and associated with the history display of FIG. 9;

FIG. 12 illustrates anatomies treated information that may be displayed and associated with the history display of FIG. 9;

FIG. 13 illustrates total number of treatments information that may be displayed and associated with the history display of FIG. 9;

FIG. 14 illustrates treatments purchased and credit units information that may be displayed and associated with the history display of FIG. 9;

FIG. 15 illustrates a dashboard display including the displays illustrated in FIGS. 7 and 9-14 according to an example embodiment of the present general inventive concept;

FIG. 16 illustrates a logged in display through which a user may update a user profile according to an example embodiment of the present general inventive concept;

FIG. 17 illustrates a user profile display according to an example embodiment of the present general inventive concept;

FIG. 18 illustrates an updatable information display for the user to edit user information according to an example embodiment of the present general inventive concept;

FIG. 19 illustrates a successful update notification display according to an example embodiment of the present general inventive concept;

FIG. 20 illustrates another example of the available treatment display of FIG. 7 according to an example embodiment of the present general inventive concept;

FIGS. 21-23 illustrate example interactive displays used to purchase treatment credits through the laser emitting apparatus according to an example embodiment of the present general inventive concept;

FIG. 24 illustrates a method of controlling the portable laser emitting apparatus according to an example embodiment of the present general inventive concept;

FIG. 25 illustrates a system to perform laser therapy including a computer system in communication with a portable laser emitting apparatus, laptop computer, and/or smartphone according to an example embodiment of the present general inventive concept;

FIG. 26 illustrates a “dashboard” display of an administrator interface used to show administrators an overview of the performance of individual users according to an example embodiment of the present general inventive concept;

FIG. 27 illustrates an “admins” display of an administrator interface used to show a list of administrators and corresponding access levels according to an example embodiment of the present general inventive concept;

FIG. 28 illustrates a “countries” display of an administrator interface used to manage sales territories according to an example embodiment of the present general inventive concept;

FIG. 29 illustrates a “distributors” display of an administrator interface used to manage sales territories by country and individual distributors according to an example embodiment of the present general inventive concept;

FIG. 30 illustrates an “email templates” display of an administrator interface used to send messages triggered by variously occurring events/factors according to an example embodiment of the present general inventive concept;

FIG. 31 illustrates a “firmware updates” display of an administrator interface used to show the latest programming updates for the laser emitting apparatuses according to an example embodiment of the present general inventive concept;

FIG. 32 illustrates a “log entries” display of an administrator interface used to show administrators laser emitting device activity according to an example embodiment of the present general inventive concept;

FIG. 33 illustrates a “models” display of an administrator interface used to show/enter model numbers of associated laser emitting devices according to an example embodiment of the present general inventive concept;

FIG. 34 illustrates an “offers” display of an administrator interface used to set increments in which treatments are offered according to an example embodiment of the present general inventive concept;

FIGS. 35A-35B illustrate an “orders” display of an administrator interface used to manage customer and administrator credited orders according to an example embodiment of the present general inventive concept;

FIG. 36 illustrates a “page templates” display of an administrator interface used to modify user dashboards according to an example embodiment of the present general inventive concept;

FIG. 37 illustrates a “price settings” display of an administrator interface used to adjust price settings on user dashboards according to an example embodiment of the present general inventive concept;

FIG. 38 illustrates a “sales representatives” display of an administrator interface used to manage sales representative data according to an example embodiment of the present general inventive concept;

FIG. 39 illustrates a “tiers” display of an administrator interface used to set prices according to numbers of treatments purchased according to an example embodiment of the present general inventive concept;

FIG. 40 illustrates a “treatments” display of an administrator interface used to edit user treatment information according to an example embodiment of the present general inventive concept;

FIGS. 41A-41B illustrate a “user devices” display of an administrator interface used to display and manage portable laser emitting device related information according to an example embodiment of the present general inventive concept;

FIGS. 42A-42B illustrate a “user devices” display of an administrator interface used to display and manage live user data according to an example embodiment of the present general inventive concept; and

FIG. 43 illustrates a method of controlling the portable laser emitting apparatus according to another example embodiment of the present general inventive concept.

### DETAILED DESCRIPTION

Reference will now be made to various example embodiments of the present general inventive concept, examples of which are illustrated in the accompanying drawings and illustrations. The example embodiments are described herein in order to explain the present general inventive concept by referring to the figures.

The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. The described progression of processing operations described are merely examples, however, and the sequence of operations is not limited to that set forth herein and may be changed as is known in the art, with the exception of operations necessarily occurring in a certain order. Also, description of well-known functions and constructions may be omitted for increased clarity and conciseness.

Various embodiments of the present general inventive concept, as described herein, provide a laser emitting apparatus that is lightweight and readily portable. In other words, the apparatus is designed so as to be easily transported from room to room, or between different locations in the same room, and therefore may be conveniently used in a home or office environment. The portable laser emitting apparatus may be used for physiotherapy and/or surgery on human and/or animal subjects, and may also have application on non-living subjects. Such various applications will be evident to one skilled in the art. In most of the various example embodiments described herein, a target area of a human subject is discussed, and the human subject may be referred to as the patient. However, it is understood that the use of laser light emitted from the portable laser emitting apparatus is not limited to such an application.

To further increase the ease of use of the portable laser emitting apparatus, according to various embodiments of the present general inventive concept, one or more laser sources of same or different wavelengths are provided in a housing of the portable laser emitting apparatus, and the laser light emitted from these laser sources is transmitted through a flexible waveguide to then be applied to a target area of the patient.

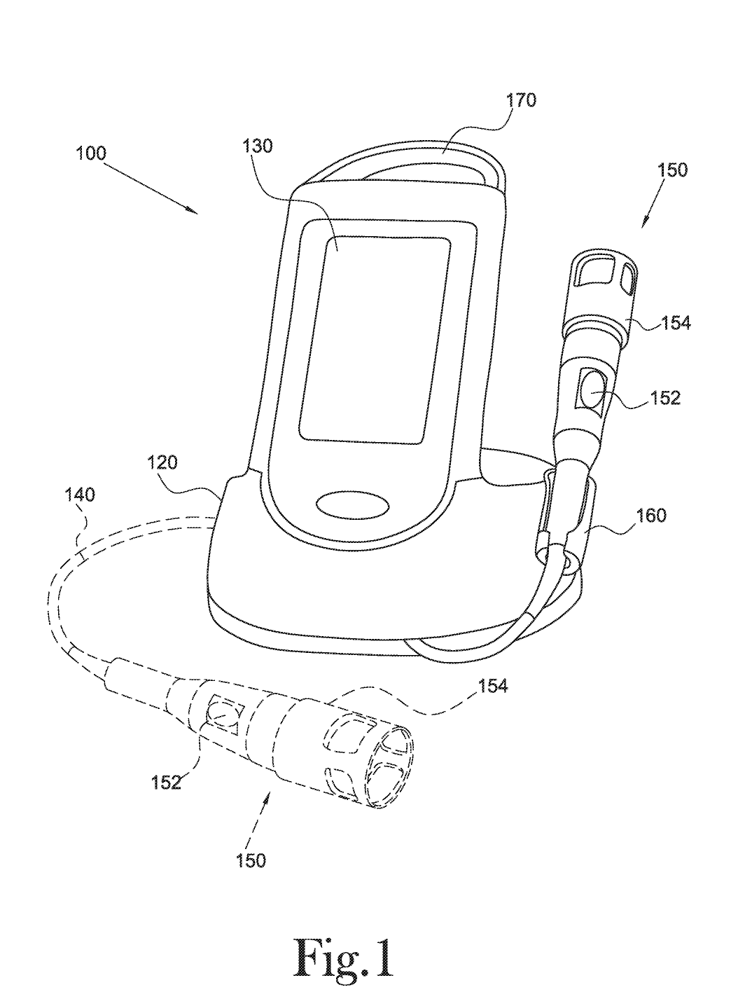

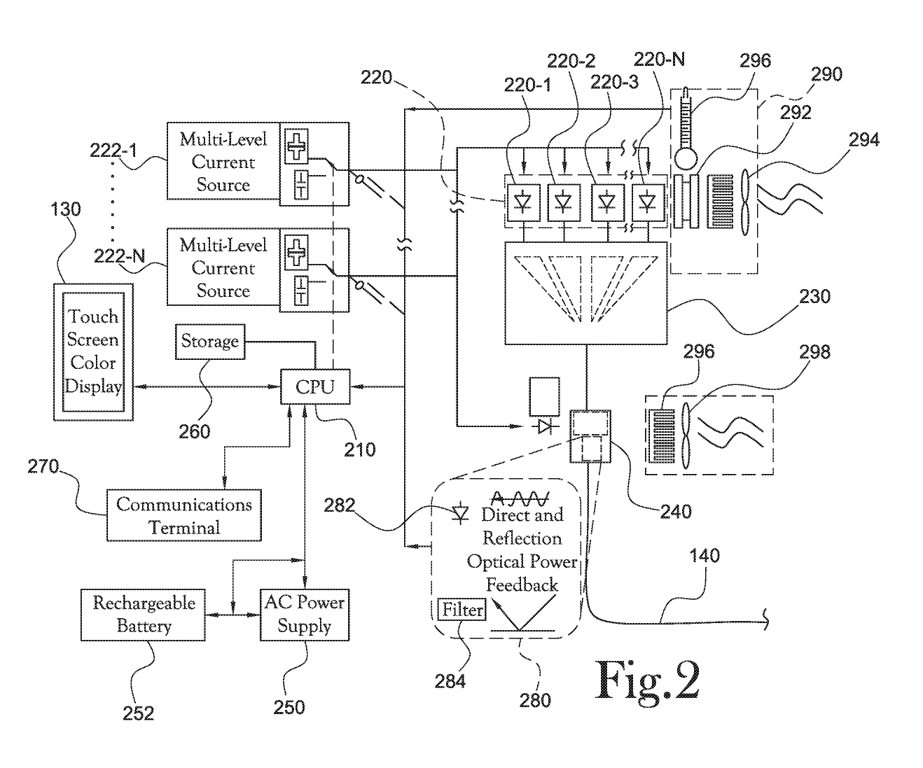

FIG. 1 illustrates a portable laser emitting apparatus according to an example embodiment of the present general inventive concept. Referring to FIG. 1, this example embodiment of the portable laser emitting apparatus 100 includes housing 120 to enclose and protect many of the components of the laser emitting apparatus 100, a touch screen display 130 to provide user interaction with the portable laser emitting apparatus, a flexible waveguide 140 to transmit laser light away from light sources provided in the housing 120 (discussed in more detail in the description of FIG. 2), and a handpiece 150 provided at a distal end of the flexible waveguide 140 to emit the laser light delivered through the flexible waveguide 140 to a target area of a patient. The example embodiment illustrated in FIG. 1 also includes a handpiece docking portion 160 which my secure the handpiece 140 when the portable laser emitting apparatus 100 is not in use, as well as a handle 170 to increase the convenience enjoyed by a user when transporting the device. To further increase the user's convenience regarding portability, in some example embodiments the portable laser emitting apparatus 100 is constructed of such lightweight material(s) as to weigh less than or equal to approximately 3.5 pounds. In other words, the portable laser emitting apparatus 100 is fabricated so as to be readily carried by a user, such as by hand, to easily move from room to room without aid from other people or mechanical aid.

It will be understood by one skilled in the art that various example embodiments of the present general inventive concept may omit various elements described in regard to the illustrated example embodiments, and that various other elements may be added. Further, the configurations of the portable laser emitting apparatus 100 are merely example configurations, and may be altered according to the various design and/or use preferences. Various components, such as the touch screen display 130, may be integrated along with the housing 120 of the portable laser emitting apparatus 100, or may be modular in order to be readily removed in the event that repair or replacement is desired.

Although not illustrated in FIG. 1, the portable laser emitting apparatus 100 may be provided with a power connection, such as an electrical cord, to be connected with an AC power source, as well as a rechargeable battery which may be provided inside or outside of the housing 120. In various example embodiments, the rechargeable battery may be removable from the portable laser emitting apparatus 100 to be recharged, and in other various example embodiments the rechargeable battery may be recharged solely by power supplied by the AC power source. In other various example embodiments of the present general inventive concept, the portable laser emitting apparatus may be equipped to receive power through wireless power transmission.

Although a touch screen display 130 is illustrated in FIG. 1, it will be understood by one skilled in the art that various other user interfaces may be employed in the portable laser emitting apparatus. For example, a conventional display and keypad combination may be substituted for the touch screen display 130. In various example embodiments, the touch screen display 130 can display images in 16 million color combinations.

The flexible waveguide 140 may be an optical cable, such as a single emitting fiber with a core size of approximately 200 um, and an NA of approximately 0.15 to 0.37. However, the waveguide 140 is not limited to an optical cable, nor an optical cable with these example attributes.

The flexible waveguide 140 may be provided with a pliable metal sheath surrounding the flexible waveguide to protect the flexible waveguide 140, and to house control wiring connected to the handpiece 150.

The example handpiece 150 illustrated in FIG. 1 is provided with at least one switch 152 to enable a user to switch between various laser application modes, and/or to start and stop emission of laser light from the handpiece 150, which will be discussed in more detail later in this description. The example handpiece 150 is also provided with a twist control 154 to enable a user to adjust the focus area of the laser application in a zoom mode, which will also be discussed in more detail later in this description. It will be understood that the at least one switch 152 and twist control 154 are merely example embodiments of the controls provided to the handpiece 150 to aid the operation by a user. Other types of controls may be added or substituted on the handpiece, or such controls may be located on the housing 120 of the portable laser emitting apparatus 100. For example, control of the laser application modes and focus areas may be controlled through the touch screen display 130 illustrated in FIG. 1.

FIG. 2 is a schematic illustration of some of the components of the portable laser emitting apparatus 100 of FIG. 1, according to an example embodiment of the present general inventive concept. According to the example embodiment illustrated in FIG. 2, the portable laser emitting apparatus 100 is provided with a CPU 210 to control the functions of the various provided components. The CPU 210 is in electrical communication with the touch screen display 130, such that commands entered by the user are then carried out by the CPU 210, and results associated with the commands, as well as other various feedback, are displayed to the user.

One or more laser sources 220 may be provided inside the housing 120. In the example embodiment illustrated in FIG. 2, multiple laser sources 220-1 through 220-N are provided inside the housing 120. However, it will be understood that any number of laser sources 220 may be provided, including a single laser source 220. The one or more laser sources 220 may have the same or different output wavelengths and/or powers. In various example embodiments, the one or more laser sources 220 may be light emitting diodes, and may be controlled to operate separately or concurrently.

In an example embodiment such as the one illustrated in FIG. 2, in which multiple laser sources 220 are provided in the housing 120, an optical combiner 230 may be provided to combine the outputs of the multiple laser sources 220 before the outputs enter the flexible waveguide 140.

The portable laser emitting apparatus may also be provided with an optical coupler 240 coupled at some point to the flexible waveguide 140. An additional laser source 242 may be provided to the optical coupler 240, and may have a wavelength between approximately 400 nm and 700 nm. The additional laser source 242 may be collimated at the emission point of the optical coupler 240, and aligned with the flexible waveguide 140. In various example embodiments, the additional laser source 242 may emit light to serve as a guiding beam to indicate an approximate point at which the energy is delivered on the target, i.e., on the tissue, and thus may function as a pointing ray, and/or in various example embodiments the additional laser source 242 may function as an additional therapeutic laser source having a biostimulating effect that may be collimated at the emission point of the optical coupler 240 and aligned with the flexible waveguide 140.

In the example embodiment illustrated in FIG. 2, the output of each of the one or more laser sources 220 are respectively controlled by corresponding one or more multi-level current sources 222-1 through 222-N. The multi-level current sources 222-1 through 222-N are controlled by the CPU 210 to adjust the output of the one or more laser sources 220 to the respective desired levels. The multi-level current sources 222-1 through 222-N may maintain the light output of the respective one or more laser sources 220 constant within 1%.

As previously discussed in regard to FIG. 1, power may be provided to the portable laser emitting apparatus 100 through an AC power supply 250, either connected to a source such as a wall adapter or received wirelessly, or through a rechargeable battery 252. According to various example embodiments, the rechargeable battery may be an internally or externally provided LiFePO4 battery, which may be removable for charging, or may be fixed and charged through the AC power supply 250.

In the example embodiment illustrated in FIG. 2, a data storage 260 is provided in electrical communication with the CPU 210 in order to store a number of preset levels to control output of the one or more laser sources 220. According an example embodiment, a total of 55 different output levels may be stored in the data storage 260, and may be easily selected by the user rather than fine-tuning the outputs of the one or more laser sources 220 to desired levels. The data storage 260 may also be used to store a control system used by the CPU 210 to control the operations of the portable laser emitting apparatus 100. Any of a number of data storage devices, such as, for example, a flash memory device, may be provided as the data storage 260.

A communication terminal 270 is also provided to the example embodiment illustrated in FIG. 2. The communication terminal may be used to provide any system and/or memory updates to the portable laser emitting apparatus 100. For example, the control system or output level presets may be updated or expanded. The communication terminal may be, for example, a USB port used to connect the portable laser emitting apparatus 100 to a computer or other electronic device to download the update data. As another example, the communication terminal may be a wireless connector, such as a Wi-Fi connector, to receive the update data in a wireless fashion.

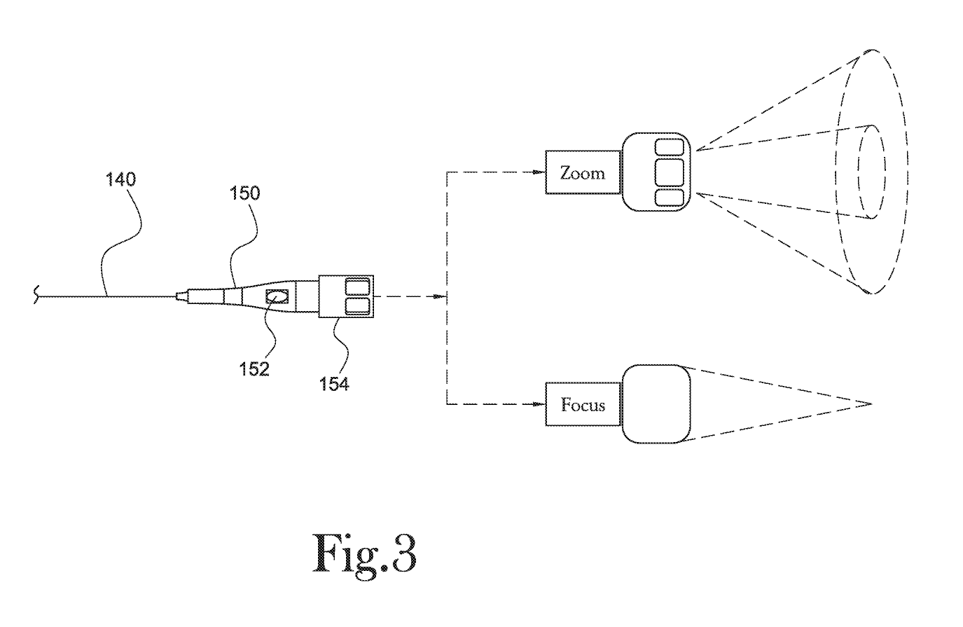

FIG. 3 illustrates example elements and functions of the handpiece 150 of the portable laser emitting apparatus 100 according to an example of the present general inventive concept. As previously described, the handpiece 150 is connected to the portable laser emitting apparatus 100 by the flexible waveguide 140. As also previously described, the handpiece 150 may be provided with at least one switch 152 to enable a user to switch between various laser application modes, and/or to start and stop emission of laser light from the handpiece 150. For example, the at least one switch 152 may be used to switch the handpiece 150 between an ON and OFF state. The at least one switch 152 may also be used to switch between focus and zoom modes of delivery of the laser light. The example handpiece 150 may also be provided with a twist control 154 to enable a user to adjust the focus area of the laser application in a zoom mode.

As illustrated in FIG. 3, the focus mode may be used to concentrate the laser light in a much smaller area of the patient, while the zoom mode may be used to spread the laser light to a comparatively larger area. In one example embodiment, the total area to which the laser light may be applied, according to the distance from the handpiece 150 to the target area, may be approximately 1.0 to 5.0 cm². The focus and zoom modes may be enable by detachable attachments to the handpiece 150, and may be mechanically or magnetically attached to the handpiece 150.

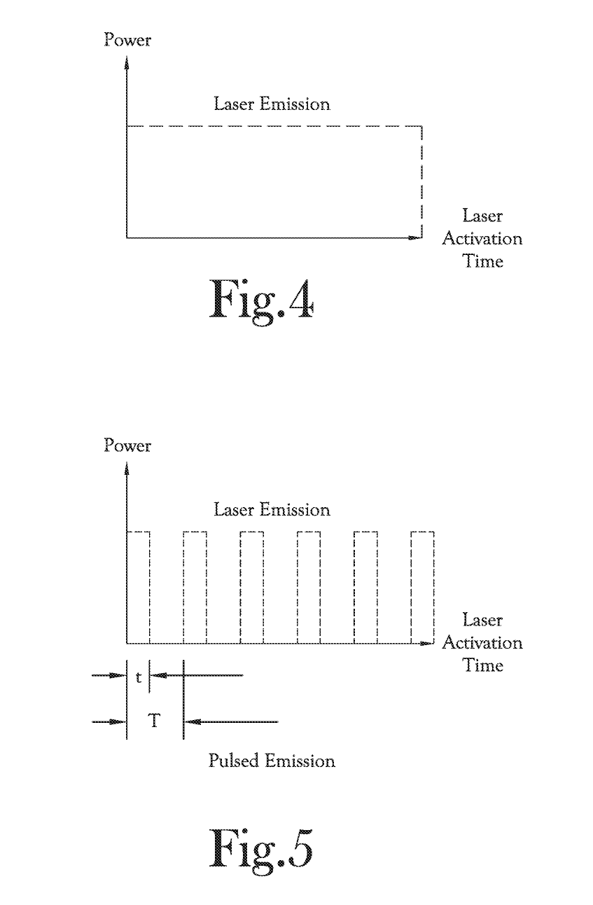

The laser emission may be applied through the handpiece 150 to the patient in several modes according to various example embodiments of the present general inventive concept. For example, according to one example embodiment, the laser light may be applied to the patient in a continuous wave mode, a frequency modulated mode, or an intense super pulse mode. These three example modes are illustrated in FIGS. 4-6.

FIG. 4 is a graph illustrating the application of the laser light in the continuous wave mode. In the continuous wave mode, the laser emission time overlaps the laser activation time, and the power of the laser emission is approximately constant.

FIG. 5 is a graph illustrating the application of the laser light in the frequency modulated, or pulsed emission, mode. In the pulsed emission mode, the laser light is only emitted in periodic pulses during the laser activation time. The laser light is emitted for a time t during each period T of the laser activation time. The duty cycle is defined as the ratio t/T, or:

Duty Cycle=t/T×100%.

The frequency of pulsed emission is the reciprocal of “T”, or:

Frequency of pulsed laser emission(Hz)=1/T.

For example, if the pulsed laser period is 1 millisecond, the frequency of pulsed laser emission would be 1 kHz.

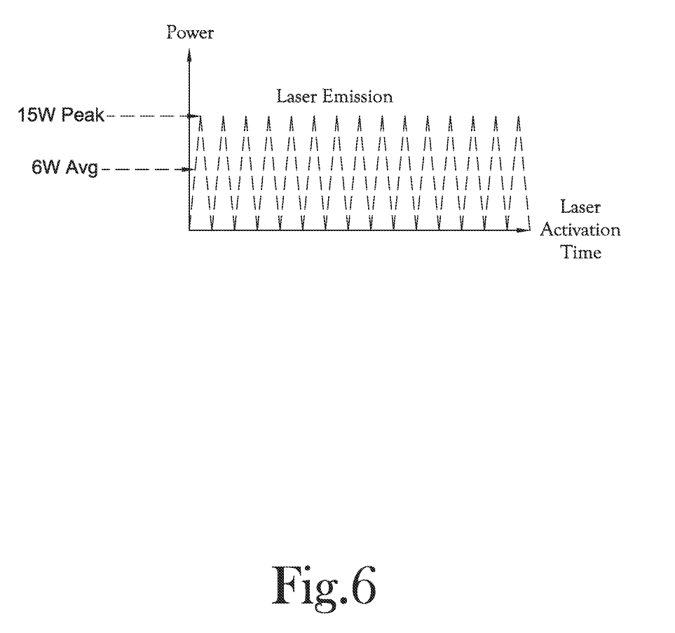

FIG. 6 is a graph illustrating the application of the laser light in the intense super pulse mode. In contrast to the continuous wave and pulsed emission modes, in which the power of the laser light is relatively constant during emission, in the intense super pulse mode the power of the laser emission is periodically and linearly adjusted between a low power and a peak power. In the example intense super pulse mode illustrated in FIG. 6, the peak power of the super pulse is approximately 15 W, while the average power output over time is approximately 6 W.

As previously discussed in regard to FIG. 2, one or more laser sources 220, having the same or different wavelengths, may be provided to the portable laser emitting apparatus 100. The one or more laser sources 220 are provided with current by the respective multi-level current sources 221 to respectively adjust the duration, intensity, and power output of the one or more laser sources 220. A few example embodiments of these one or more laser sources 220, configured and operated accordingly to better treat various medical conditions, will now be described.

In one example embodiment of the present general inventive concept, the portable laser emitting apparatus is provided with a first laser source 220-1 and a second laser source 220-2. The first laser source 220-1 may have a wavelength of 660 nm, with a power output of up to 100 mW, which is known to have stimulatory effects in superficial dermatological conditions such as open wounds, diabetic ulcers and infections. The second laser source 220-2 may have a wavelength of 800 nm, with a power output ranging from 0.1 to 8 or 12 Watts. This second laser source 220-2 is in the NIR range and is centered at the peak of cytochrome c oxidase's absorption.

Another example embodiment may include the first and second laser sources 220-1,220-2 described above, along with a third laser source 220-3, which may have a wavelength of 970 nm, with a power output ranging from 0.1 to 8 or 12 Watts. This wavelength is also in the NIR range and is centered at the peak of water's absorption. This third laser source 220-3 may create thermal gradients on the cellular level along which blood would more readily flow.

Yet another example embodiment may include the first through third laser sources 220-1,220-2,220-3 described above, along with a fourth laser source 220-4, which may have a wavelength of 905 nm, with a power output ranging from 0.1 to 8 or 12 Watts. This fourth laser source 220-4 sits at the peak of oxy-hemoglobin's absorption, thereby increasing the flow of oxygen from the blood to the cells for processing. It should be apparent to one skilled in the art that these are only some of the examples of useful laser wavelengths, and various other embodiments of the present general inventive concept may have any number of other combinations of laser sources, wavelengths, and/or powers. In other words, the present general inventive concept is not limited to the several examples described above.

According to various example embodiments, normal frequency modulated operation, such as that illustrated in FIG. 5, may provide a frequency range adjustable from 1 to 20,000 Hz with an average power ranging from 0.1 to 6 Watts. In various example embodiments of the intense super pulse mode, the frequency range may remain adjustable from 1 to 20,000 Hz, but the peak power may be 21 Watts, with the average power being adjustable from 0.1 to 8 Watts.

The example embodiment illustrated in FIG. 2 also includes various safety features. One such feature is the luminous emission feedback system 280. Included in this luminous emission feedback system 280 is a photodiode 282 and a special direct and reflection optical power system filter 284. Through this filter 284, the photodiode 282 can detect the luminous emissions coming from the irradiated tissues of the patient, which can occur at different wavelengths. These luminous emissions are proportional to the absorption and temperature increase in the patient caused by the emission of laser light from the handpiece 150. The data from the luminous emission feedback system 280 is transmitted to the CPU 210 for analysis and system control. In particular, the luminous emission feedback system 280 insures the efficacy of the optical treatment and maintains the safety level high and constant. Further, this luminous emission feedback system 280 allows an automatic check on the intensity of the laser light delivered to the tissue to optimize the therapeutic effects while guaranteeing maximum safety. Still further, this luminous emission feedback system 280 can gather information for eventual diagnostic purposes.

Another example safety feature in the example embodiment illustrated in FIG. 2 is the use of a cooling system 290 for the one or more laser sources 220. For example, a peltier cooler 292 and fan 294 may be provided to the one or more laser sources 220. Both the peltier cooler 292 and fan 294 may be electrically connected to and under the control of the CPU 210. A temperature gage 296 may be mounted on the one or more laser sources 220 and connected to the CPU 210 so that the CPU 210 can monitor and control the temperature of the one or more laser sources 220 through the control of the peltier cooler 292 and fan 294. This is merely one example through which a safe temperature of the one or more laser sources 220 may be maintained, and is not intended to limit the ways that one can control and maintain proper temperature of the laser sources. One skilled in the art would be aware of other methods for controlling the laser sources in a safe operating temperature range.

A further safety feature illustrated in the example of FIG. 2 is through the providing of a heat sink 296 and a fan 298 on the optical coupler 240, which ensures that the optical coupler 240 remains at a safe operating temperature.

According to various example embodiments of the present general inventive concept, a number of analysis and/or control operations such as, for example, treatment tracking, revenue estimation, credit-based activation, and the like may be available to the user of the portable laser emitting apparatus 100 through interaction with a remotely located device management system. The remote device management system may be a supplier of the portable laser emitting apparatus 100, and/or may track usage information and control various operations of the apparatus 100 as described herein. It is understood that “remote device management system” is simply a term to describe the remotely located managing entity of these services, and is typically an automated management process performed by a processor and memory configuration such as one or more networked server computers, or servers, or similar information processing device, e.g., computer, that is able to communicate data to and from the apparatus 100 through, for example, an internet or cellular connection. In various example embodiments herein, the remote device management system may be referred to as a computer system to manage a remote laser emitting apparatus used in medical treatments.

In various example embodiments, a graphical user interface (GUI) may be displayed on the touch screen display 130 to allow a user of the laser emitting apparatus 100 to track treatments that have been performed and/or will be performed in the future, to estimate current and future revenue based on past and projected future treatments, and/or to perform various other operations that may also be displayed on other electronic communication devices communicating with the remote device management system, such as a smartphone, laptop computer, and the like. The tracked information may be shared with the remote device management system, and the revenue estimates may be provided by the remote device management system, by online or other similar communications between the devices. In various example embodiments, the portable laser emitting apparatus 100 may be authorized to be operated on a credit-based system, wherein the user purchases, through the GUI, a certain number of procedures or time of operation of the device from a remote control location. In various example embodiments, the GUI may be displayed on the touch screen display 130, or may be accessed on any other processing device having a display, such as a computer or smartphone. When displayed on a device other than portable laser emitting apparatus 100, the GUI may be controlled by the CPU 210 of the portable laser emitting apparatus 100 through the communications terminal 270. In other various example embodiments, the GUI may be controlled by, or include information submitted from, other locations, such as a remote site for controlling access to the laser emitting apparatus 100. In other words, various examples of displays describe herein may be displayed on, for example, a laptop computer that is in communication with the remote device management system which is providing the display information. In such an example embodiment, the laptop computer may also be communicating with the portable laser emitting apparatus 100 at the same time, or the portable laser emitting apparatus 100 may receive updated information from the remote device management system or the laptop computer (or other information processing device) at a later time. It is understood that the terms “display” and “GUI” may be used interchangeably in the descriptions of these various example embodiments. The CPU 210 of the laser emitting apparatus 100 may communicate with the remote device management system at the remote site through a variety of ways, such as, for example, a wired internet connection, a wireless connection to a home network, a cellular based connection, and so on.

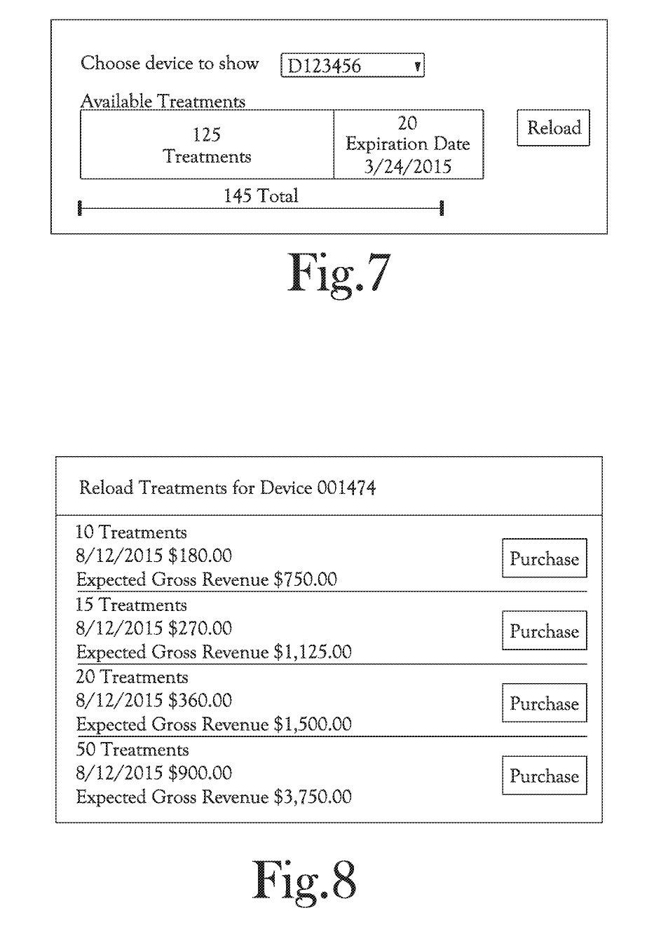

According to various example embodiments of the present general inventive concept, the remote device management system may control a display, to be displayed on a user's device, that presents several categories of information that may be viewed by the user, and which may be selected to display further information according to the selected category. The collection of display information may be referred to herein as a “dashboard” display, and may be the initial display shown to a user upon the user logging in to the remoted device management system's user interface. FIGS. 7-14 illustrate a few example categories/displays that may be displayed in the dashboard display. The dashboard features described herein for access by the user of the laser emitting apparatus may be referred to as user dashboard features, which may be part of the user dashboard, to differentiate from dashboards and/or dashboard features typically accessed by remote device management system administrators.

FIG. 7 illustrates an available treatments display used to track and control operations of the portable laser emitting apparatus according to an example embodiment of the present general inventive concept. The available treatments display illustrated in FIG. 7 may be included in the initial display shown when a user accesses either the laser emitting apparatus 100 itself, or a web site operated by the remote device management system at a remote location controlling credit-based operations of the laser emitting apparatus 100. In FIGS. 7-20, the illustrated displays or portions of displays are shown as being accessed through a web page provided by the remote device management system, such as would be displayed on a laptop computer or the like, but it is understood that the same or similar information may be displayed on the touch screen display 130 of the portable laser emitting apparatus 100, and may be provided to the apparatus 100 by the remote device management system. The system for purchasing credits from the remote device management system at the remote location may be referred to as an on demand system, and such language may be used to refer to the system in the description of various example embodiments described herein. The remotely located entity that controls the credit-based operations and other various assorted data management operations may be referred to in descriptions herein as the remote device management system of the portable laser emitting apparatus operations, or simply the remote device management system.

In various example embodiments, a user may generate, or be provided with, a username and password that are associated with the user's account with the remote device management system. For example, the username and password may be requested by, and provided through, an email exchange with the remote device management system. The user may then access the remote device management system's web page and log into the user's account with the username and password. In various example embodiments, the user may log in and access information from the remote device management system using the touch screen display 130 of the laser emitting apparatus 100.

In various example embodiments, a dashboard display may be displayed that is concentrated in a plurality sections selectable by a user such as, for example, available treatments, expected revenue, history, statistics, etc. The layout of the dashboard display will be described in more detail later in the description of FIG. 14. These sections may be selected by the user upon logging in to access the user's account. For example, the available treatments display illustrated in FIG. 7 allows the user to choose, from a dropdown menu, any devices associated with the user, in the event that more than one device is registered to the user. In the case in which the user is only associated with one device, e.g., one laser emitting apparatus 100, the identification information of that device may simply be displayed without the dropdown menu option. In FIG. 7, the available treatments display shows that the currently displayed device associated with the identification code D123456 has 145 total available treatments, and that 20 of the 145 available treatments are going to expire by Sep. 24, 2015. Therefore, the user has previously made arrangements with the remote device management system to purchase at least 145 treatments, some of which have an expiration date by which they must be used. For example, in some example embodiments the remote device management system assigns purchased credits a 30 day window in which the credits should be used, or the credits may expire at the end of the 30^{th} day. The remaining 125 available treatments indicated in FIG. 7 may also have expiration dates associated therewith, but are not as close to expiring as the 20 treatments for which the expiration date is shown. A reload button may be provided in the available treatments display illustrated in FIG. 7 for the user to purchase additional treatments. In various example embodiments, the different areas in the bar graph indicating the available treatments may be color coded to differentiate between treatments that are and are not in danger of expiring. For example, the treatments that are not close to expiration may be displayed in green, while the treatments that are nearing expiration may be displayed in red. In various example embodiments, if a user clicks the reload button to add additional treatments to the user's account, a display may be triggered to show the user different predetermined purchase options from which the user may choose.

FIG. 8 illustrates a purchase options display used to allow a user to choose from a plurality of purchase options according to an example embodiment of the present general inventive concept. In various example embodiments, if a user selects the reload button illustrated in FIG. 7, the display in FIG. 8 may be displayed to allow the user different predetermined credit package options from which to choose. The purchase options display may be, for example, a pop-up window that is only displayed when the user selects the reload button. As indicated by the example embodiment illustrated in FIG. 8, the user may choose to purchase the treatment credits in quantities of 10, 15, 20, and 50. In various example embodiments, the purchase options display may include incentive information indicating free credits that may be earned by purchasing treatment credit packages of higher quantities. In some example embodiments, the user may be provided with an entry window to enter a different quantity than those shown in the predetermined packages.

In various example embodiments, the newly purchased treatments may be added to the displayed information the next time the user synchronizes the laser emitting apparatus 100 with the remote device management system. In some example embodiments, the laser emitting apparatus 100 may automatically attempt to synchronize with the remote device management system upon the passage of a predetermined time increment. In other example embodiments, the newly purchased treatments may be instantaneously added to the apparatus 100 by the remote device management system communicating with the communications terminal 270 of the apparatus 100, and authorized with the apparatus 100 itself upon the next synchronization or other such communication between the remote device management system and the laser emitting apparatus 100.

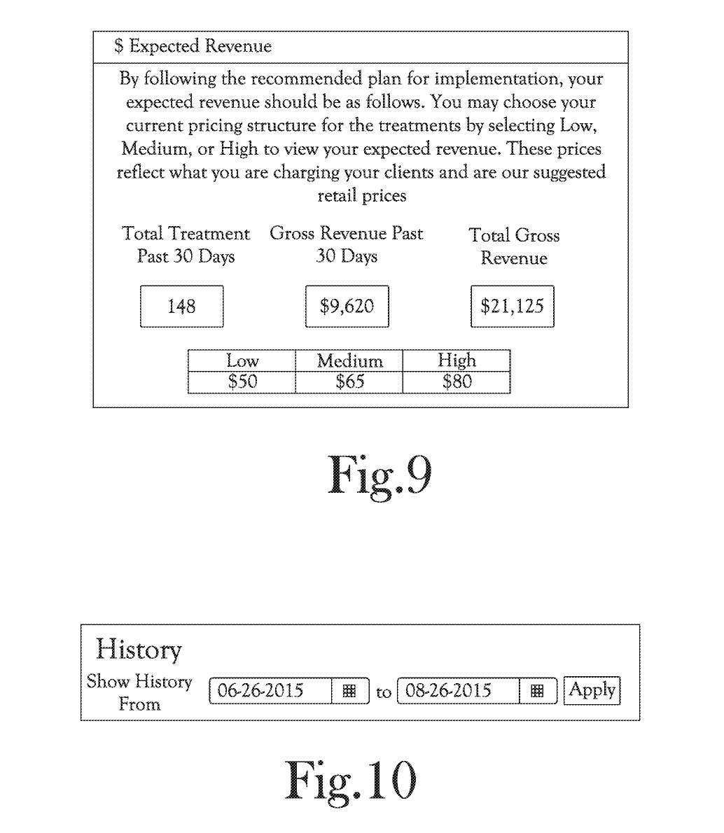

FIG. 9 illustrates an expected revenue display to track and control operations of the portable laser emitting apparatus according to an example embodiment of the present general inventive concept. As illustrated in FIG. 9, the total number of treatments performed for the current 30 day cycle may be displayed along with the gross revenue from the associated cycle. The total gross revenue for the life of the device may also displayed. In various example embodiments, the expected revenue display may advise the user that “By following the recommended plan for implementation, your expected revenue should be as follows. You may choose your current pricing structure for the treatments by selecting Low, Medium, or High to view your expected revenue. These prices reflect what you are charging your clients and are our suggested retail prices.” The Low, Medium, and High estimation of charges may be based on a national average of, for the purpose of this example, $75 a treatment. The number of total treatments performed during a current cycle may be reset every 30 days. The Low, Medium, and High estimations in FIG. 9 may be selectable by the user to show the corresponding revenue totals so that a user may make a more informed decision when determining what price to charge for treatments. In the example illustrated in FIG. 9, the gross revenue for the 148 treatments is based on the selected Medium price of $65. The user may select from the Low and High buttons on the display to see the corresponding differences in those charges per 148 treatments.

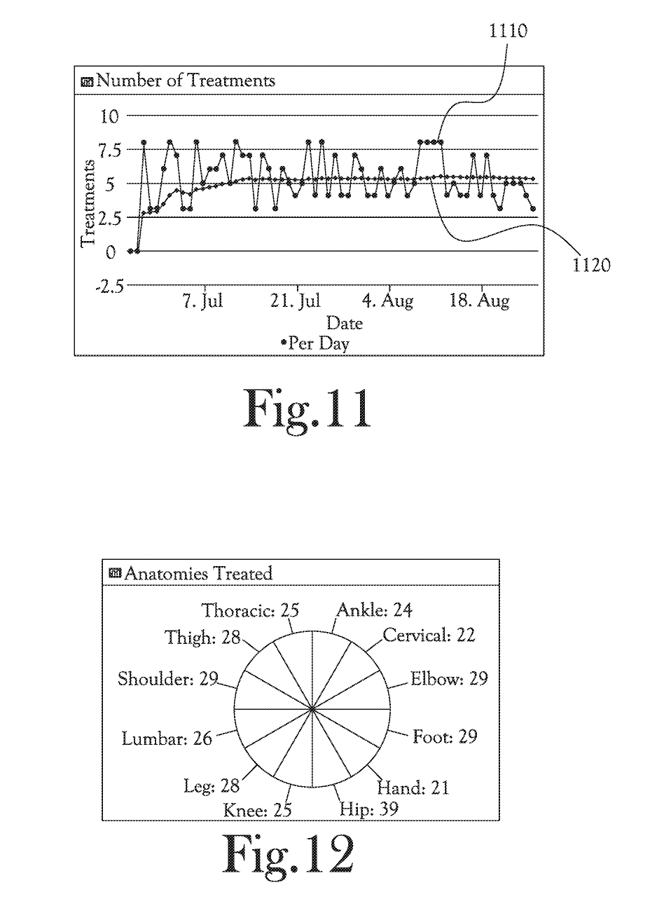

FIG. 10 illustrates a history display used to track and control operations of the portable laser emitting apparatus according to an example embodiment of the present general inventive concept. As illustrated in FIG. 10, the user may be able to select dates, either by, for example, direct alphanumeric entry or through dropdown calendar menus, that define a range for which the user wishes to see associated device information. For example, the associated device information may include a number of treatments performed with the laser emitting apparatus 100, and/or the anatomical regions (or anatomies) treated, during the selected range. FIG. 11 illustrates number of treatments information that may be displayed and associated with the history display of FIG. 10. As illustrated in FIG. 11, the information may be plotted as a number of treatments on the vertical axis, and the date range that was entered by the user on the horizontal axis. The line graph 1110 indicates the total number of treatments per day of the selected time range, and the line graph 1120 indicates the average number of treatments per day over 30-day cycles. In various example embodiments, the user can move a cursor over particular dots of the line graphs to see the exact numbers associated with the selected dots. FIG. 12 illustrates anatomies treated information that may be displayed and associated with the history display of FIG. 10. As illustrated in FIG. 12, the information may be shown in a pie graph, wherein each piece of the pie graph shows the number of treatments performed on an associated portion of the anatomy. The pie chart illustrates a breakdown of anatomies treated by the user during the specified time frame. In various example embodiments, the user can move a cursor over each piece of the pie chart to view the exact number of times the user has treated that particular anatomy. In various example embodiments, the user will enter such anatomy information periodically, such as when a treatment is performed, and the data will be collected and organized by the remote device management system so that the user can access an easy visual representation of the data at his/her leisure. In some example embodiments, the user may enter the anatomical information for desired treatments when purchasing treatment credits, so that the remote device management system may assign treatment time ranges to the treatment credits such that more specific treatment times of use are set according to the desired treatments. For example, treatments for hip or lumbar regions may require longer time than those for fingers or toes, and thus the treatment time per credit can be adjusted accordingly by the remote device management system.

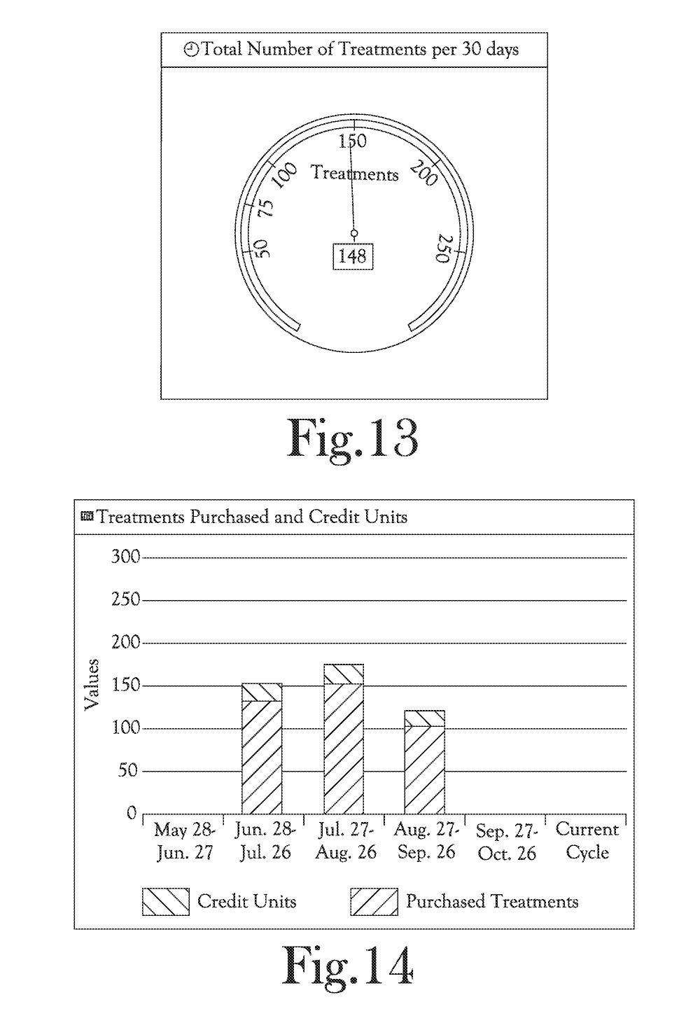

FIG. 13 illustrates total number of treatments information that may be displayed and associated with the history display of FIG. 10. In the example embodiment of FIG. 13, the display is based on a current 30-day cycle of the user, which is indicated as a number on a dial, which may be referred to as a speedometer, with the actual number displayed in the center of the dial. In example embodiments, the display in FIG. 13 is relative to the number of treatment credits applicable to the current 30-day cycle, i.e., the period of time at the end of which one or more of the treatment credits may expire. FIG. 14 illustrates treatments purchased and credit units information that may be displayed and associated with the history display of FIG. 10. In the example embodiment of FIG. 14, the display is a bar chart indicating treatments purchased and credit units. The terms “credit units” and “treatment credits” may be used interchangeably herein. As the user loads the portable laser emitting apparatus 100 with purchased treatment credits, the purchased credits may show up on the bar chart of FIG. 14 with a color or shade indicated below the chart. These purchased treatment credits may be located in the bottom portion of each respective bar of the bar chart. The treatment units that are otherwise credited to the user's account by the remote device management system as, for example, incentive rewards, may be shown on the bar chart with a different color or shade, as also indicated below the chart, and which may be located in the top portion of the respective bars of the bar chart. These credited treatment units may be earned, for example, by purchasing a specific amount of treatment credits during a 30-day cycle or during the initial pre-load of the device. In other words, the purchased treatments indicated in FIG. 14 were actually purchased by the user, while the credit units were credited to the user's account through, for example, an incentive rewards program. In various example embodiments, a user's continued participation in the on demand program may be dependent on the number of credits the user purchases during a 30-day cycle. For example, the user may be provided the laser emitting apparatus for a reduced charge, or no charge, if the user commits to purchasing a certain threshold number of treatment credits every 30 days. Failure to meet these threshold requirements could result in the user having to forfeit the apparatus, or pay a penalty, and so on. These GUI tools aid the user, as well as the remote device management system, in keeping track of such purchases.

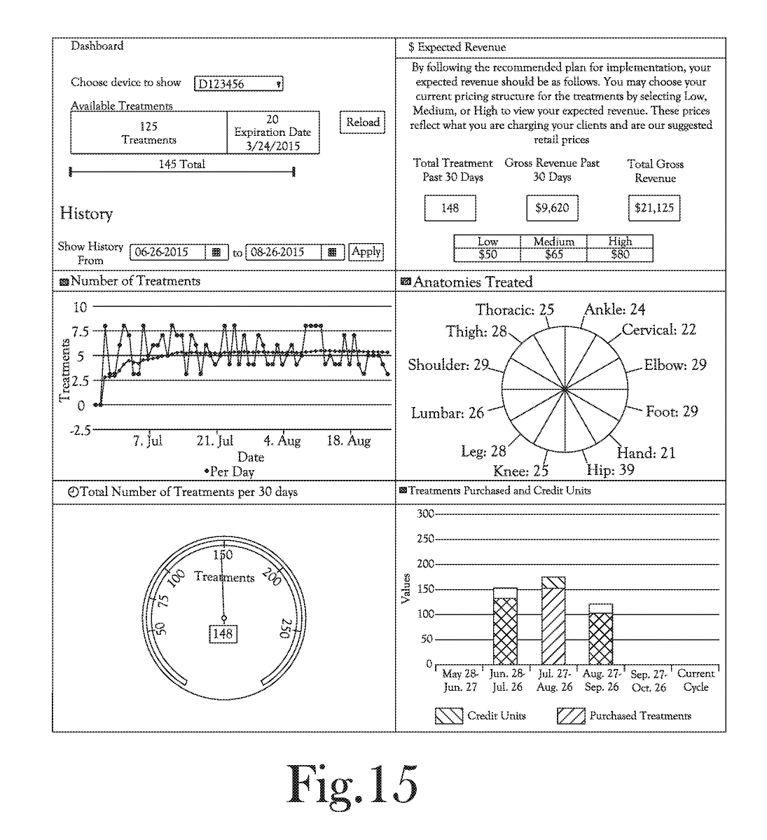

FIG. 15 illustrates a dashboard display including the displays illustrated in FIGS. 7 and 9-14 according to an example embodiment of the present general inventive concept. In this example embodiment, the illustrated dashboard includes all of the previously described displays made available to the user, absent the pop-up menu that results from the user choosing the reload button. It is understood that different example embodiments may have different areas of information, different configurations, and so on, for the user to choose from. Also, the dashboard display may be available in a mobile version for users to access from a device such as a smartphone. In the mobile version of the dashboard display, one or more areas of the display may be represented as a clickable option on the display or a drop-down menu, and the corresponding information may be displayed on a different “page” when the user selects that option.

In various example embodiments of the present general inventive concept, the user may be provided with a user profile through which the remote device management system may securely store user information associated with a corresponding device, payment, and so on. The user information may include general personal information such as name, address, state, phone number, etc., along with the device identification information corresponding to the user information. For example, the user information may include the user's billing information corresponding with a serial number of the corresponding laser emitting apparatus 100. The user may be responsible for keeping such billing information up to date and making any necessary changes to ensure uninterrupted on demand service from the remote device management system. The user account may be initiated by the user sending user information to the remote device management system, for example, by email, instant messaging, and/or interacting with the remote device management system interface at a web page administered by the remote device management system, at which point the user profile may be set up in the system. Once the user confirms the user account, in various example embodiments the user may easily view and edit information through remote device management system interface, which may be referred to as the on demand dashboard, at any time.

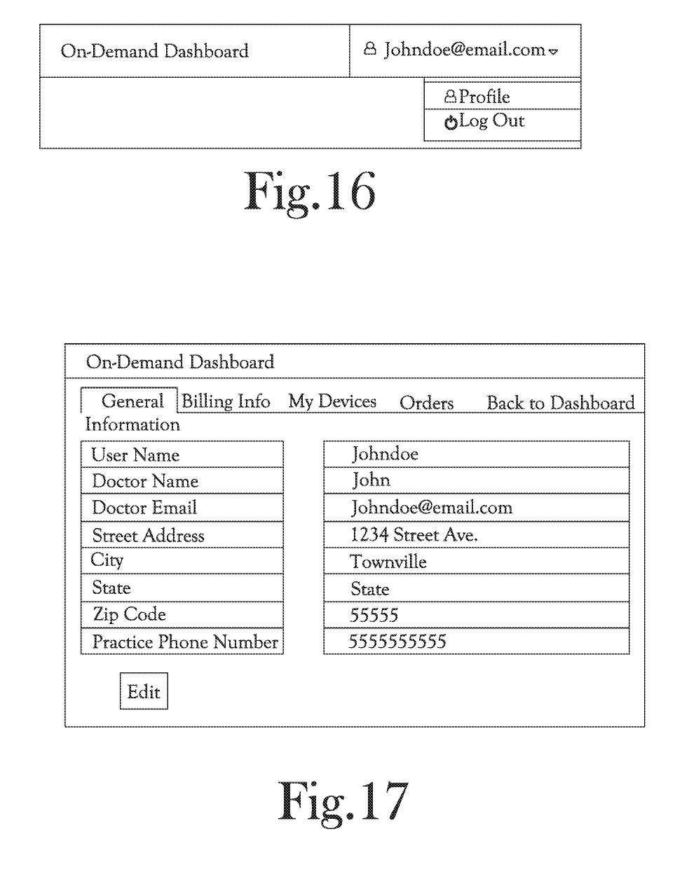

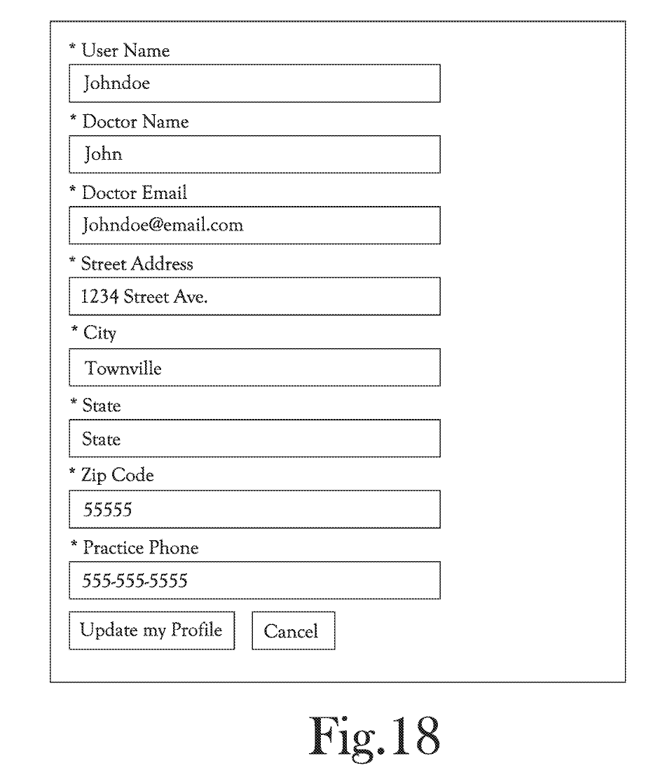

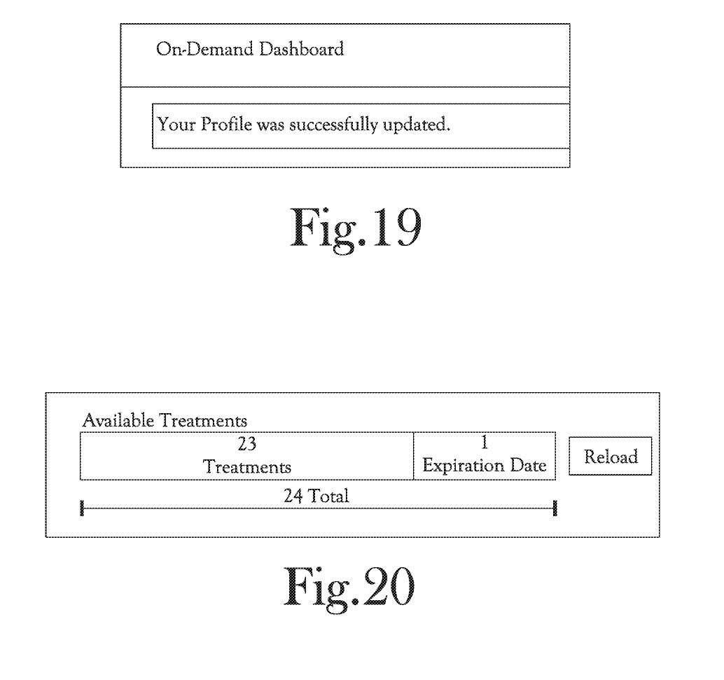

In various example embodiments, the user can update the user information by going to an on demand web site of the remote device management system and log in with the user's username and password that has been previously established. FIG. 16 illustrates a logged in display through which a user may update a user profile according to an example embodiment of the present general inventive concept. The display of FIG. 16 indicates that the user is logged in by showing the user's identification (an email address in this example) and a log out option. In this example embodiment, the user simply clicks on “Profile” to bring up profile information to be edited or verified. FIG. 17 illustrates a user profile display according to an example embodiment of the present general inventive concept. In the example embodiment shown in FIG. 17, the user may simply click the tab of the type of information that the user wishes to edit, such as general information, billing information, devices, orders, etc. One or more fields may then be displayed according to the chosen tab. In the example embodiment of FIG. 17, the user has chosen the General Information tab, and corresponding fields for user name, doctor name, doctor email, and practice phone number are displayed. The user may click the Edit button to edit one or more of the displayed fields. FIG. 18 illustrates an updatable information display for the user to edit user information according to an example embodiment of the present general inventive concept. In the example embodiment of FIG. 18, the user can simple change any of the information displayed in the text fields to update the corresponding information, and then select the Update My Profile button to save the changed data. FIG. 19 illustrates a successful update notification display according to an example embodiment of the present general inventive concept. In the example embodiment of FIG. 19, the user is notified by text that the user profile has been successfully updated. In various example embodiments, the text notifying the user of the successful update may be located in a color-coded box having, for example, a green background, to further indicate that update was successful.

In various example embodiments of the present general inventive concept, as previously discussed, a user may add treatment credits to the corresponding laser emitting apparatus 100 through the on demand website discussed above, and/or directly through the laser emitting apparatus 100. FIG. 20 illustrates another example of the available treatment display of FIG. 7 according to an example embodiment of the present general inventive concept. In order to purchase treatment credits through the on demand portal provided by the remote device management system, after logging in the user may simply choose the available treatments display. In various example embodiments, the available treatments display may be included in the initial on demand dashboard display, or may be accessed by the user by clicking an available treatments button. As previously described, the user may then click the reload button, which may be color-coded to aid in quick identification by the user, next to the bar chart displaying the number of available treatments. In some example embodiments, a pop-up display such as that illustrated in FIG. 8 may be displayed to allow the user to select from various predetermined packages of treatment credits. According to various example embodiments, upon selecting the reload button the user may be prompted to simply enter the number of treatments the user would like to purchase. In various example embodiments, the treatments may be available in packages of 5, 10, 20, 50, and so on. Certain quantities may qualify the user for free credit units as discussed previously in regard to FIG. 14. After choosing the desired quantity, the user may execute the transaction, for example, by clicking a purchase button which authorizes the remote device management system to bill the user accordingly. In various example embodiments, the purchase may be further confirmed by another confirmation button that the user must click after clicking the purchase button. In various example embodiments, the treatment credits may be added to the user's account and visible in the remote device management system's on demand dashboard instantly, and purchased credits may appear and be available for use on the user's laser emitting apparatus 100 after the apparatus 100 has communicated with the remote device management system, for example, by a synchronization over a Wi-Fi or other such connection. The user may also be provided with a confirmation email, text message, etc., that includes the purchase details corresponding to the transaction.

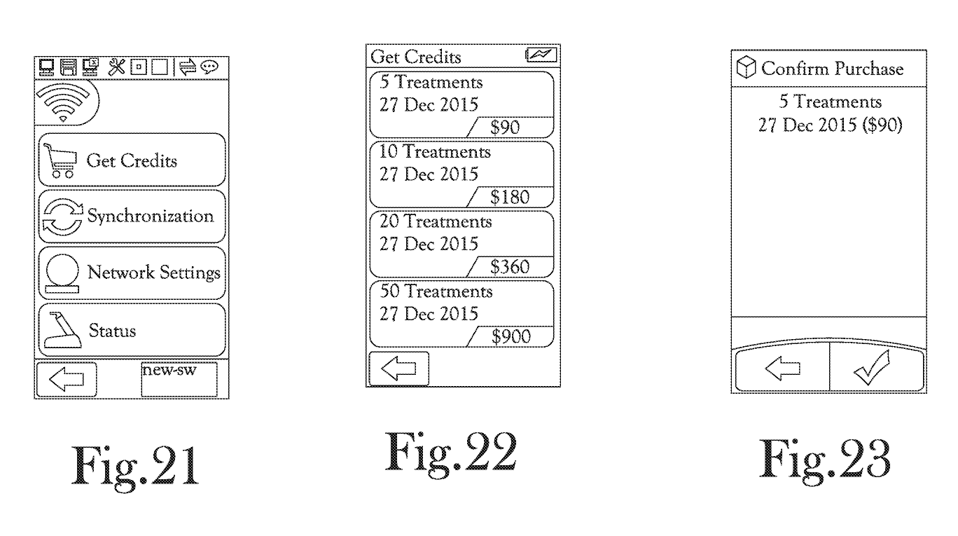

In various example embodiments of the present general inventive concept, the user may be able to purchase treatment credits from the remote device management system directly through the portable laser emitting apparatus 100, which may also be referred to as an on demand device. In various example embodiments, the apparatus 100 may need to be in communication with the remote device management system during the purchase, such as through a wired or wireless internet connection, a cellular connection, or the like. FIGS. 21-23 illustrate example interactive displays used to purchase treatment credits through the laser emitting apparatus according to an example embodiment of the present general inventive concept. Upon turning the laser emitting apparatus 100 on using the on/off button, the user may be prompted to enter a PIN code or other such identification information that may be used to unlock various processes of the apparatus 100. In example embodiments in which the entry of the PIN code is used, upon entry the home screen of the apparatus 100 may be displayed on the touch-screen display 130. FIG. 21 illustrates an example embodiment of the home screen of the apparatus 100. From the home screen of FIG. 21, the user may select several operations such as, for example, Get Credits, Synchronization, Network Settings, Status, and so on. To purchase treatment credits, the user may select the Get Credits button. In various example embodiments, the touch-screen display 130 will display one or more available treatment packages, as illustrated in FIG. 22. The treatment credit packages illustrated in FIG. 22 include quantities of 5, 10, 20, and 50, along with the corresponding prices and expiration dates for each package. The user may select the treatment package that the user desires to purchase by selecting the corresponding button on the touch-screen display 130. According to various example embodiments, the user may then be presented with the display shown in FIG. 23, which displays the treatment credit package selected by the user, along with the corresponding price and expiration date, and the user is able to confirm the purchase by selecting the appropriate button on the screen. The apparatus will then communicate with the remotely located remote device management system, either immediately because of the purchase by the user, or at a later time when either the user synchronizes the device with the remote device management system or when the remote device management system initiates a communication, so that the device will be authorized to use the newly purchased treatment credits. According to various example embodiments, the remote device management system may send an email, text message, or the like to the user to memorialize the purchase details. Such details may also be displayed by the apparatus 100 following a communication with, and an authorization by, the remote device management system.

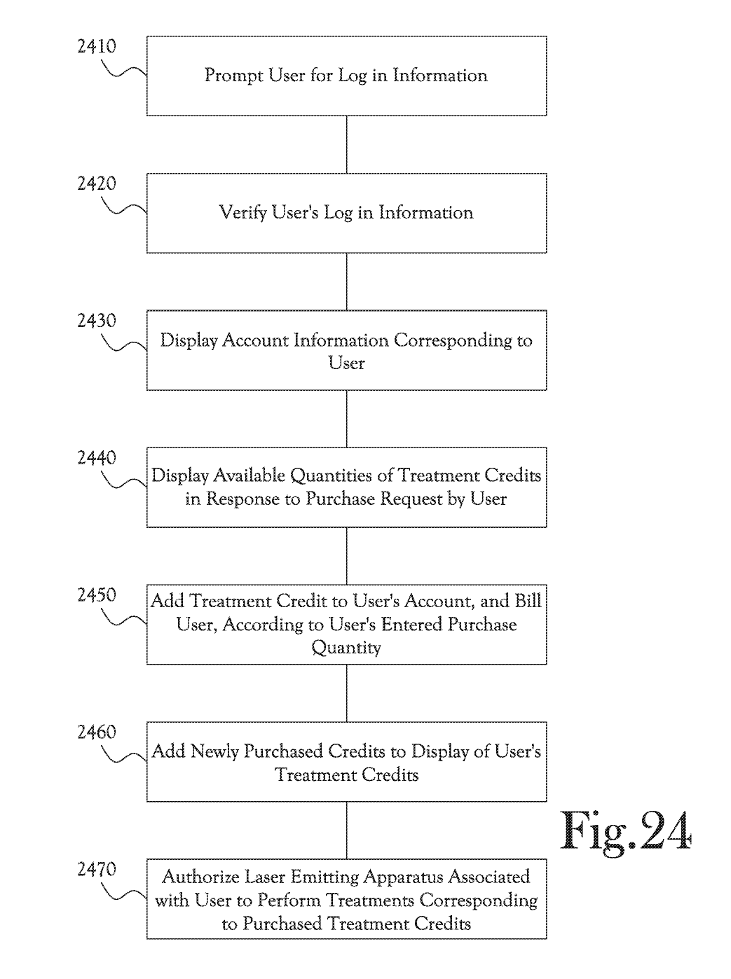

FIG. 24 illustrates a method of controlling the portable laser emitting apparatus according to an example embodiment of the present general inventive concept. It is understood that no specific order is implied for all of the operations illustrated in FIG. 24, and various other example embodiments may include more or fewer operations. Additionally, the example embodiment illustrated in FIG. 24 may include operations not explicitly listed in FIG. 24, and/or sub-operations that correspond to the listed operations. When an interface such as the previously discussed on demand dashboard is accessed by a user, e.g., when a computer system to manage a remote laser emitting apparatus used in medical treatments is contacted by the user through, for example, a web page, the computer system, which may be otherwise referred to as the remote device management system, may prompt the user for log in information in operation 2410. When the user's log in information has been entered, the remote device management system may verify the log in information by comparing the entered log information with information previously stored in one or more data storages of the remote device management system in operation 2420. Once the user's log in information is verified, the remote device management system may display the user's account information in operation 2430, through which the user can edit information, review treatment history, etc., as well as purchase treatment credits. In the event that the user indicates a desire to purchase treatment credits, the remote device management system may display available quantities of treatment credits to the user in operation 2440. In operation 2450, the remote device management system may add the treatment credits to the user's account, and bill the user, according to the desired purchase quantity entered by the user. The remote device management system may add the newly purchased treatment credits to the display of the user's total number of treatment credits in operation 2460. In operation 2470, the remote device management system may authorize the portable laser emitting apparatus associated with the user through the user's account information to perform the number of treatments corresponding to the number of purchased treatment credits. The authorized number of treatments may include any previously purchased treatment credits with the newly purchased treatment credits, according to whether any of the treatment credits have expired due to use and/or expiration dates. According to various example embodiments, all of the purchased and otherwise obtained treatment credits may have a corresponding expiration date associated therewith.

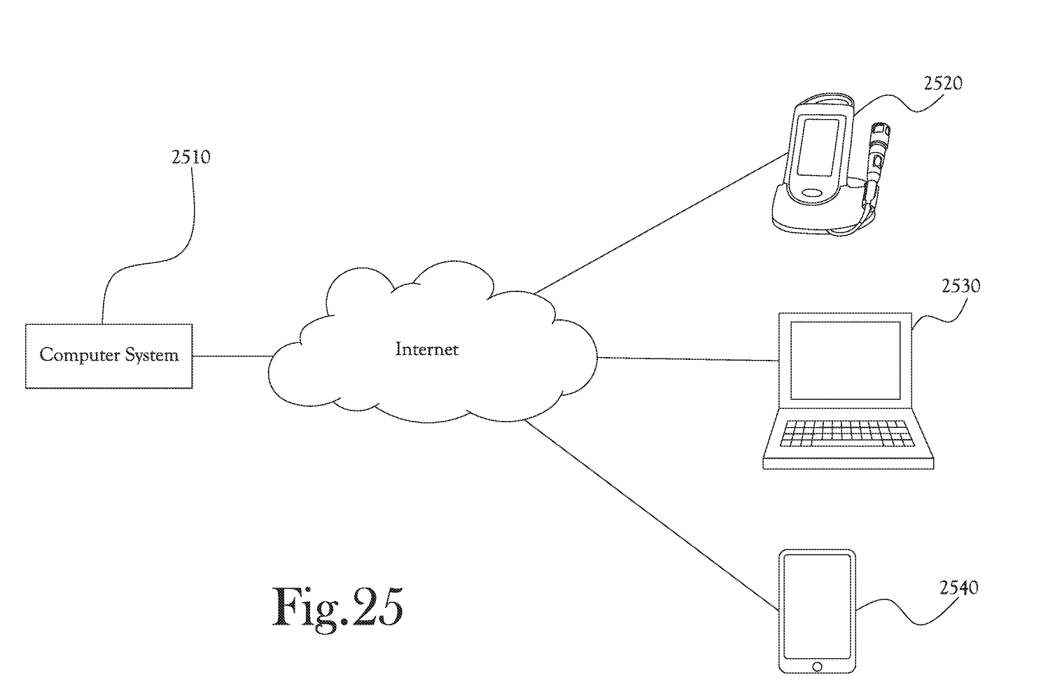

FIG. 25 illustrates a system to perform laser therapy including a computer system in communication with a portable laser emitting apparatus, laptop computer, and/or smartphone according to an example embodiment of the present general inventive concept. It is understood that any of a number of electronic communication devices with displays may be used to communicate with the computer system managing the laser emitting apparatus, and that the system to perform laser therapy is not limited to those items illustrated in FIG. 25. In the example embodiment illustrated in FIG. 25, the computer system 2510 may communicate with the laser emitting apparatus 2520 through a communication path such as the internet, which may also be used to communicate with the laptop computer 2530 and the smartphone 2540. As previously described, in other various example embodiments the computer system 2510 may communicate with the illustrated devices through a cellular communication connection. In various example embodiments, the laser emitting apparatus 2520, laptop computer 2530, and/or smartphone 2540 may communicate with one another or the computer system 2510 through a Wi-Fi connection.

According to various example embodiments of the present general inventive concept, a method of using a computer system to manage a remote laser emitting apparatus used in medical treatments may include the computer system receiving, through computer data communication, apparatus identification information associated with a remotely located laser emitting apparatus, and user identification information associated with a user of the laser emitting apparatus, the computer system storing the apparatus identification information and user identification information, the computer system correlating, in response to receiving data regarding a purchase of one or more treatment credits by the user, the purchased treatment credits to the laser emitting apparatus associated with the user, and the computer system communicating the purchased treatment credits to the laser emitting apparatus through computer data communication, wherein the purchased treatment credits authorize the laser emitting apparatus to perform a number of treatments corresponding to the purchased treatment credits. The computer data communication may be communicated via the internet. The data regarding the purchase of the one or more treatment credits by the user may be received from the laser emitting apparatus. The data regarding the purchase of the one or more treatment credits may be entered directly into the laser emitting apparatus by the user. The method may further include the computer system controlling a user interface so as to be displayed on a remote device, wherein the data regarding the purchase of the one or more treatment credits by the user is facilitated through the user interface. The user interface may be a web page displayed on an information processing device. The method may further include the computer system prompting the user for the user identification information at an initial display of the user interface. The method may further include the computer system displaying in the user interface a total number of treatments for which the laser emitting apparatus is currently authorized. The method may further include the computer system displaying in the user interface an expiration date by which one or more of the corresponding authorized treatments must be performed. The method may further include the computer system displaying in the user interface an option for the user to purchase additional treatment credits. The method may further include the computer system displaying in the user interface various components indicating expected revenue based on data collected from the laser emitting apparatus through computer data communication. The method may further include the computer system displaying in the user interface historical data regarding usage of the laser emitting apparatus according to a date range entered by the user through the user interface. The method may further include the computer system communicating with the laser emitting apparatus through computer data communication to compile the historical data. The historical data may include at least one of treatment dates or anatomies treated. The method may further include the computer system displaying in the user interface statistical data regarding laser emitting apparatus usage and/or treatment credits based on one or more monthly cycles.

According to various example embodiments of the present general inventive concept, a portable laser emitting apparatus to be used in physiotherapy and/or surgery may be provided, the apparatus including a readily portable housing, one or more laser sources of same or different wavelengths provided in the housing, a flexible waveguide extending from the housing and configured to transmit laser light from the one or more laser sources to a target area, a data storage configured to store a plurality of predetermined settings of wavelength and power combinations to be emitted from the one or more laser sources, data corresponding to past usage of the laser emitting apparatus, and treatment credit information regarding whether the laser emitting apparatus is authorized to perform one or more treatments, a communication terminal configured to receive data updates for the controller and/or data storage from a remote device management system, and a controller configured to control the one or more laser sources to operate according to the predetermined settings in response to the laser emitting apparatus being authorized to perform a treatment, the past usage data, and communication with the remote device management system to share the usage data and/or receive new treatment credits. The controller may be configured to perform a quantity of treatments that corresponds to a number of treatment credits stored in the data storage. The treatment credits may be communicated to the laser emitting apparatus from the remote device management system. The portable laser emitting apparatus may further include a touch screen display configured to display usage information and to receive treatment credit purchase information from a user.

According to various example embodiments of the present general inventive concept, a non-transitory storage medium may be provided having recorded thereon machine readable instructions to cause a computer system to perform a method of managing a remote laser emitting apparatus used in medical treatments, the method including the computer system receiving, through computer data communication, apparatus identification information associated with a remotely located laser emitting apparatus, and user identification information associated with a user of the laser emitting apparatus, the computer system storing the apparatus identification information and user identification information, the computer system correlating, in response to receiving data regarding a purchase of one or more treatment credits by the user, the purchased treatment credits to the laser emitting apparatus associated with the user, and the computer system communicating the purchased treatment credits to the laser emitting apparatus through computer data communication, wherein the purchased treatment credits authorize the laser emitting apparatus to perform a number of treatments corresponding to the purchased treatment credits.

According to various example embodiments of the present general inventive concept, a system to perform laser therapy may be provided, including a portable laser emitting apparatus configured to perform laser therapy, and a remotely located computer system configured to receive, through computer data communication, apparatus identification information associated with the laser emitting apparatus, and user identification information associated with a user of the laser emitting apparatus, to store the apparatus identification information and user identification information, to correlate, in response to receiving data regarding a purchase of one or more treatment credits by the user, the purchased treatment credits to the laser emitting apparatus associated with the user, and to communicate the purchased treatment credits to the laser emitting apparatus through computer data communication, wherein the purchased treatment credits authorize the laser emitting apparatus to perform a number of treatments corresponding to the purchased treatment credits.

While the embodiments of the remote device management system and various related components and operations discussed to this point have generally focused on a single user accessing the remote device management system to purchase treatment credits to be used with the laser emitting apparatus associated with the user, and to access other information related to the laser emitting apparatus, various example embodiments of the present general inventive concept may provide a method of using a computer system to manage a plurality of remote laser emitting apparatuses used in medical treatments, and computer systems with which to implement the method. Information/data associated with the plurality of users, or clients, and corresponding user devices may be accessed and/or controlled by administrators of the remote device management system. Such administrators may access and/or control this information through a displayed interface, in a similar manner that the users may access an on demand interface such as, for example, the user dashboard previously described. The on demand interface that is controlled by the remote device management system, and through which program administrators of the remote device management system may monitor and control the plurality of devices and associated data, may be referred to as the on demand administration interface, and may be used to monitor and control any number of devices in a plurality of locations, including international locations. In various example embodiments of the present general inventive concept, the on demand administration interface may be the central interface for program administrators to manage users, treatments, devices, etc. Several example embodiments and features of an on demand administration interface will be described in relation to the drawings herein. It is understood that the terms “administrator” and “program administrator” may be used interchangeably, and simply refers to a person who is authorized to access and control certain types of information through the on demand administration interface. As will be discussed herein, different administrators may have different levels of access and/or control. While the on demand administration interface and system is designed for use by international distributors as well as domestic ones, the functions of a United States administrative interface are mainly represented in the drawings herein for the sake of simplicity. The data managed by the various administrators is organized/stored such that administrators from various locations can access the data and manipulate the data as needed and/or authorized. Portions of the data may be directly accessed by an administrator on a local area network, from a remotely stored location through a web based application, and so on.

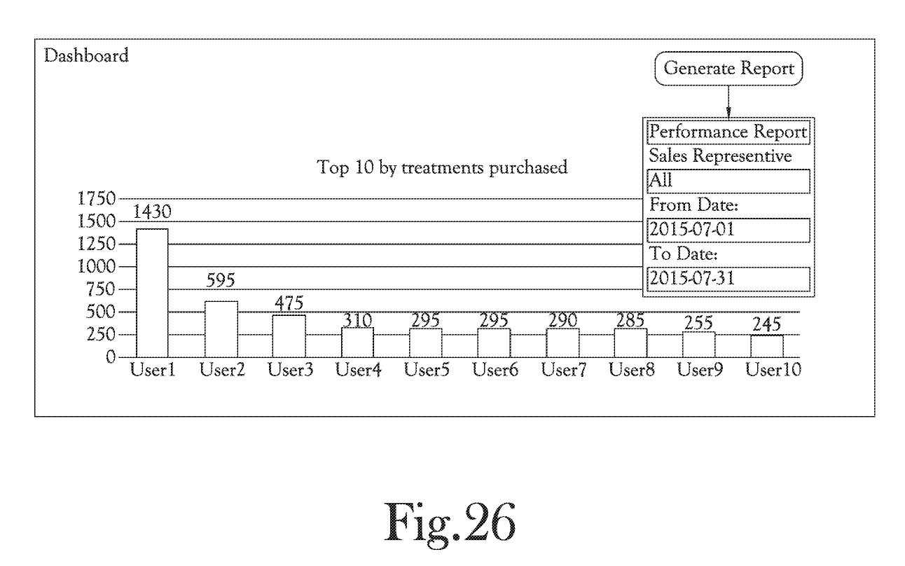

FIG. 26 illustrates a “dashboard” display of an administrator interface used to show administrators an overview of the performance of individual users according to an example embodiment of the present general inventive concept. The example embodiment of the dashboard display illustrated in FIG. 26 gives administrators a quick overview of the performance of individual users and a detailed look at the program's financial health by individual user 30 day treatment cycles or fiscal/calendar dates. Different example embodiments may be configured to show different information. Such a dashboard display may be set as the default display that is shown after an administrator has successfully logged in to the remote device management system through the administrator interface. In the example embodiment illustrated in FIG. 26, a bar graph shows the top ten users by treatments purchased, indicating the number of treatments purchased by each of the top ten users. The top ten users are referred to as User1, User2, and so on in the illustration of FIG. 26, but may have the users' registered usernames or other identifying characteristics in an actual display. Although not illustrated in FIG. 26, another bar graph may be displayed that shows the lowest 10 users by treatments purchased. The lowest 10 users graph may be displayed on the same screen, the administrator may scroll down to see the second graph, the administrator may select a button to access the second graph, or other similar methods of displaying the information may be employed. Another available graph may be the 10 users with the most expired treatments. Various example embodiments may display a host of easily categorized information so that administrators may get a quick and easy overview of different points of interest as related to the users and users' activities. Although bar graphs are illustrated in FIG. 26, other types of graphs may be used, or altogether different types of displays of data. Different sections of the graphs may be color coordinated to aid in quick understanding of the information. According to various example embodiments, the administrator may prompt the system to generate a performance report based on sales representatives. Such a procedure may be actuated by choosing a button displayed on the dashboard display, and such a button may include a label such as “Generate Report”. The example embodiment of FIG. 26 illustrated various fields that may be completed by the administrator to prepare such reports. For example, the administrator may be prompted to enter the names of one or more sales representatives, or “All” the sales representatives, and start and end dates for the performance report. Other time periods, such as commissions based on 30-day treatment cycles, may be chosen instead of start and end dates. Such performance reports may be displayed in, for example, an .xls format for easy viewing by the administrator.

FIG. 26, and many of the drawings to follow, simply illustrate portions of a display that may be shown to an administrator according to various example embodiments of the present general inventive concept. The entirety of the screen of the various displays has not been illustrated for the sake of clarity and easy understanding of these various features. It is understood that the displays described herein may include more or less information, and may be presented in a variety of ways, without departing from scope of the present general inventive concept. For example, at a top portion of each of the displays discussed herein, a plurality of buttons may be displayed that allow the administrator to go straight to any one of the displays described herein. In various example embodiments, the plurality of buttons may be labeled, for example, as Dashboard, Admins, Countries, Distributors, Email Templates, Firmware Updates, Log Entries, Models, Offers, Orders, Page Templates, Price Settings, Sales Representatives, Suggested Prices, Tiers, Treatments, User Devices, and Users. The Dashboard display has been described in the discussion of FIG. 26. The other listed displays will be described in more detail in the discussion of many of the following drawings.

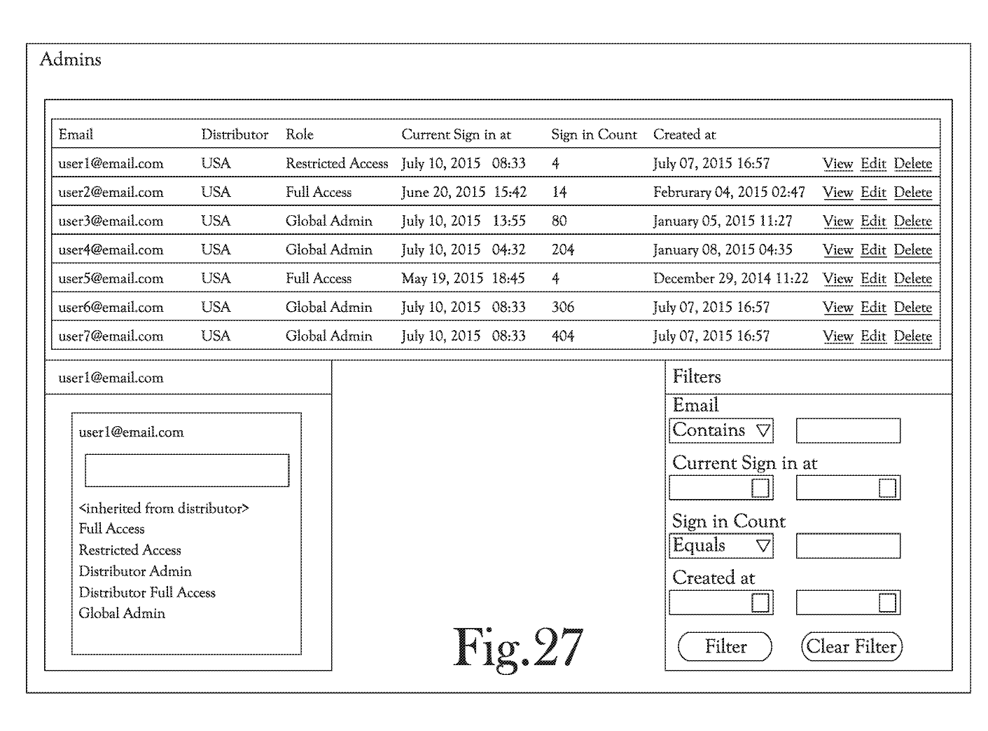

FIG. 27 illustrates an “admins” display of an administrator interface used to show a list of administrators and corresponding access levels according to an example embodiment of the present general inventive concept. The admins display of this example embodiment allows a user (in this case an administrator, not and end user of the portable laser emitting apparatus) to manage different levels of administrator access to application data in the remote device management system. As illustrated in FIG. 27, the administrators may be listed by some type of identification data such as an email address, and the information displayed may include each administrators' associated distributor, access level (listed as “role” in this example embodiment), current sign in date and time, sign in count, and creation date. According to various example embodiments of the present general inventive concept, different access levels may include “global admin”, having full rights access to all functions/features of the application (including control over distributors and admins); “full access”, having full rights access to all functions/features of the application; “restricted access”, having access restricted to viewing information only; “distributor full access”, having access to all amenities of a distributor interface, and limited in scope to users and devices assigned to that distributor; and “distributor admin”, including access that is restricted to viewing information. It is understood that these are simply different access levels of this example embodiment of the present general inventive concept, and other example applications may include different access levels, both in quantity and authorization. The view/function of the admin panel and the admins interface may be modified based on the level of access of the administrator using the admins page (or display). According to various example embodiments, a “new admin” button may be displayed to allow the administrator user to enter the corresponding information for a newly registered administrator. Also, as illustrated in FIG. 27, a filters window may be displayed to allow the administrator user to filter the results of the admins display. For example, the administrator user may adjust the searched for characters used in the email addresses displayed, the current sign in field, the sign in count, the creation date, and so on. Such filtering allows the administrator user to narrow the number of administrators for which the corresponding information is displayed in the admins display. Another window illustrated in FIG. 27 allows the administrator user to adjust the role of other administrators, assuming the administrator user has the access level required to do so. In other words, the admins page allows an authorized administrator user to manage different levels administrator access to application data. It is understood that the word “page” in the context of these descriptions may be used interchangeably or instead of “screen”, as the described pages are display screens displayed in various GUI's on various devices.

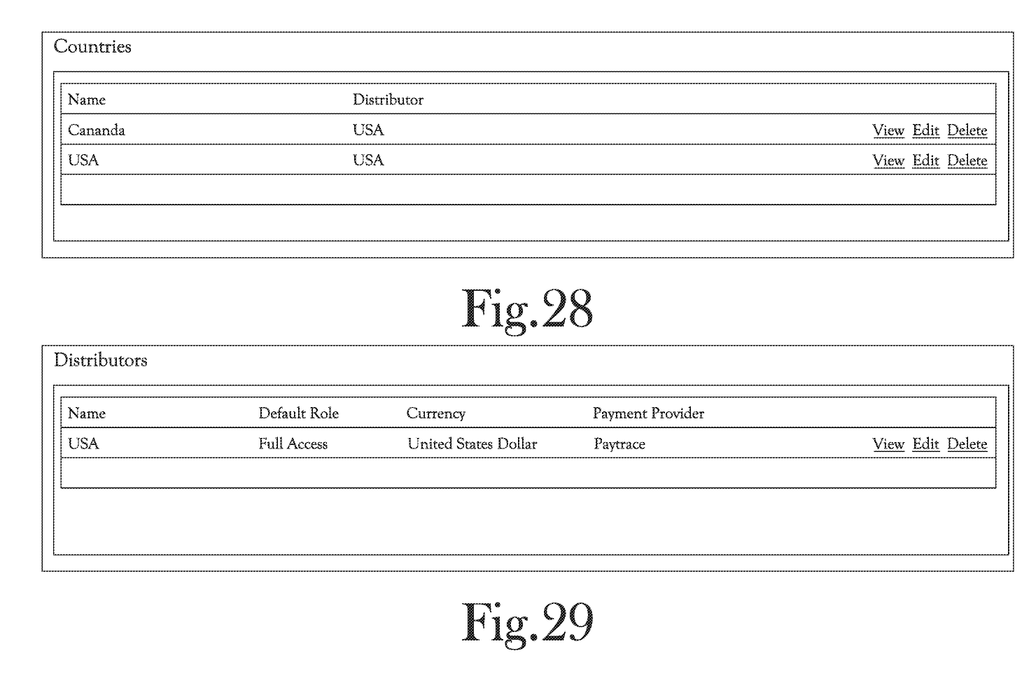

FIG. 28 illustrates a “countries” display of an administrator interface used to manage sales territories according to an example embodiment of the present general inventive concept. The countries display in the example embodiment illustrated in FIG. 28 allows administrators to manage sales territories within the program by country. For example, the distributor “USA” may refer to portable laser emitting apparatuses and users managed by the United States distribution territories. New distributors may be entered by, for example, company names. As with many of the fields in this and the previously described administrator interface pages, view, edit, and delete buttons may be displayed to allow the administrator to alter the associated data displayed on the page. It is understood that the terms “page” and “display” may be used interchangeably throughout these descriptions.

FIG. 29 illustrates a “distributors” display of an administrator interface used to manage sales territories by country and individual distributors according to an example embodiment of the present general inventive concept. According to various example embodiments, the distributors page may only be accessed by administrators having a global administrator access level, which allows the administrators to manage sales territories within the program by country and individual distributors. Various functions of the distributors display may include allowing administrators to create, edit, and delete distributors, determine the distributor overall access rights level, set the local currency type, assign the distributor payment processing company to interface with the secure web application programming interface (API), and so on.

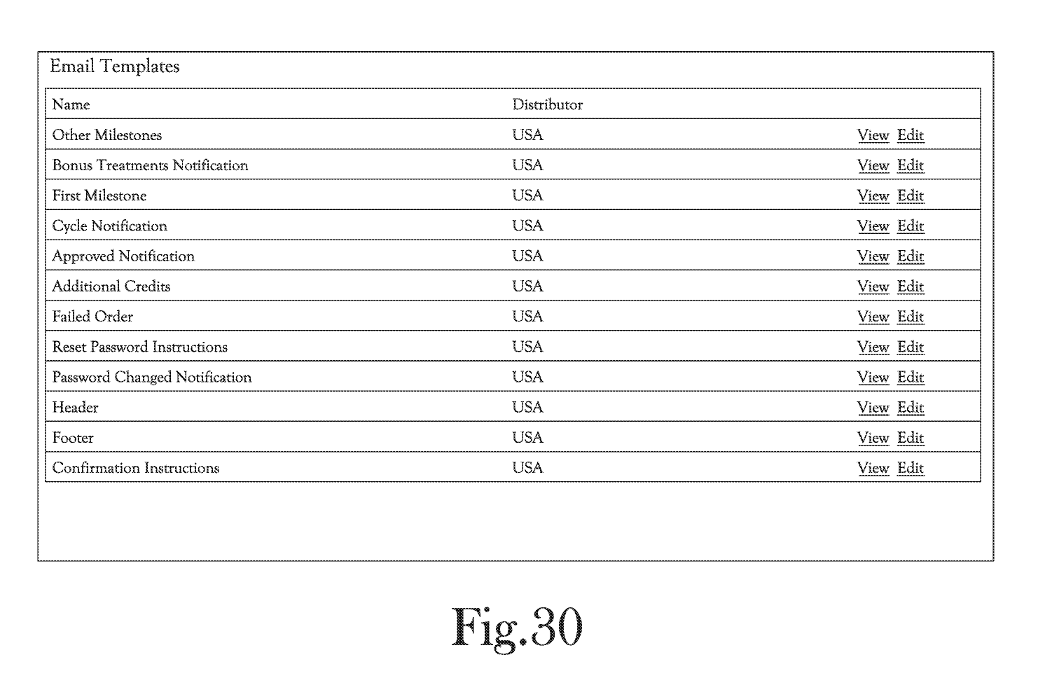

FIG. 30 illustrates an “email templates” display of an administrator interface used to send messages triggered by variously occurring events/factors according to an example embodiment of the present general inventive concept. The email templates selectable by the administrator may be used to send real-time messages to users and select administrators based on, for example, distributors and triggers within the API. This allows an administrator to quickly and easily send out emails for various expected or recurring events. Examples of emails and their corresponding triggers illustrated in FIG. 30 may include “other milestones”, for certain quantities (e.g., 100, 150, 200, etc.) of treatments purchased; “bonus treatments notification”, for a certain number (e.g., 75) of treatments purchased; “first milestone”, for a certain number (e.g., 50, which may be a baseline monthly goal) of treatments purchased; “cycle notification”, sent a certain number of days (e.g., 15, 5) before a 30-day cycle ends; “approved notification”, for when a user is approved by an administrator; “additional credits”, for when, for example, bonus credits are sent to a user; “failed order”, for when an order was not processed correctly; “reset password instructions”, for when a user forgets his/her password; “password changed notification”, for when the user's password is changed; “header”, to change header logo/content (by distributor); “footer”, to change footer content (by distributor); and “confirmation instructions”, for a user to confirm the user's account. Such email templates allows an administrator to quickly communicate messages associated with various expected and/or recurring instances without starting “from scratch” each time when preparing the communication.

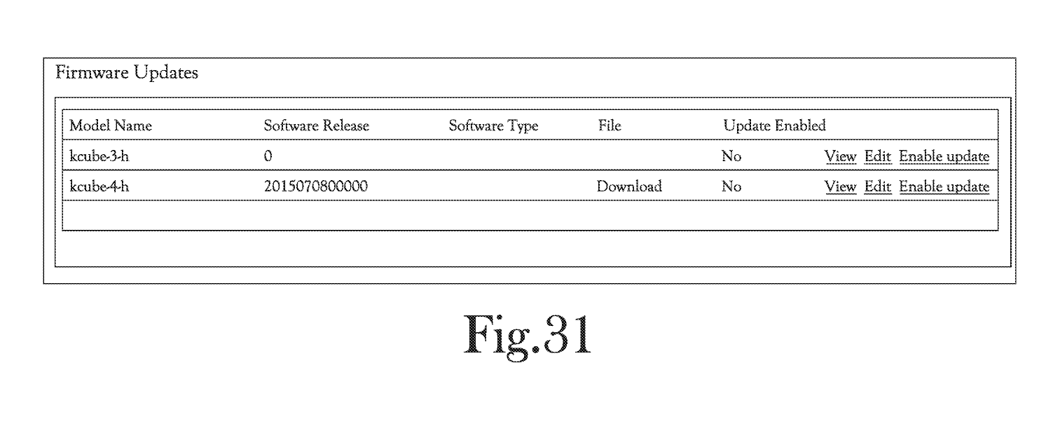

FIG. 31 illustrates a “firmware updates” display of an administrator interface used to show the latest programming updates for the laser emitting apparatuses according to an example embodiment of the present general inventive concept. In the example embodiment illustrated in FIG. 31, the latest software releases may be posted by the administrators to the firmware updates page for the various laser models used in the program. These firmware updates may be transmitted to the laser emitting apparatuses so that they may always be operating on the latest firmware. A message may be broadcast to the on demand laser emitting apparatus units via the web API and Wi-Fi device internet connection when an update is enabled. The update may also be downloaded by administrators for the purpose of sending the file to a user for a manual update via, for example, USB flash drive, if needed. With such a display, an administrator may easily see what firmware updates have become available, and the time at which they became available.

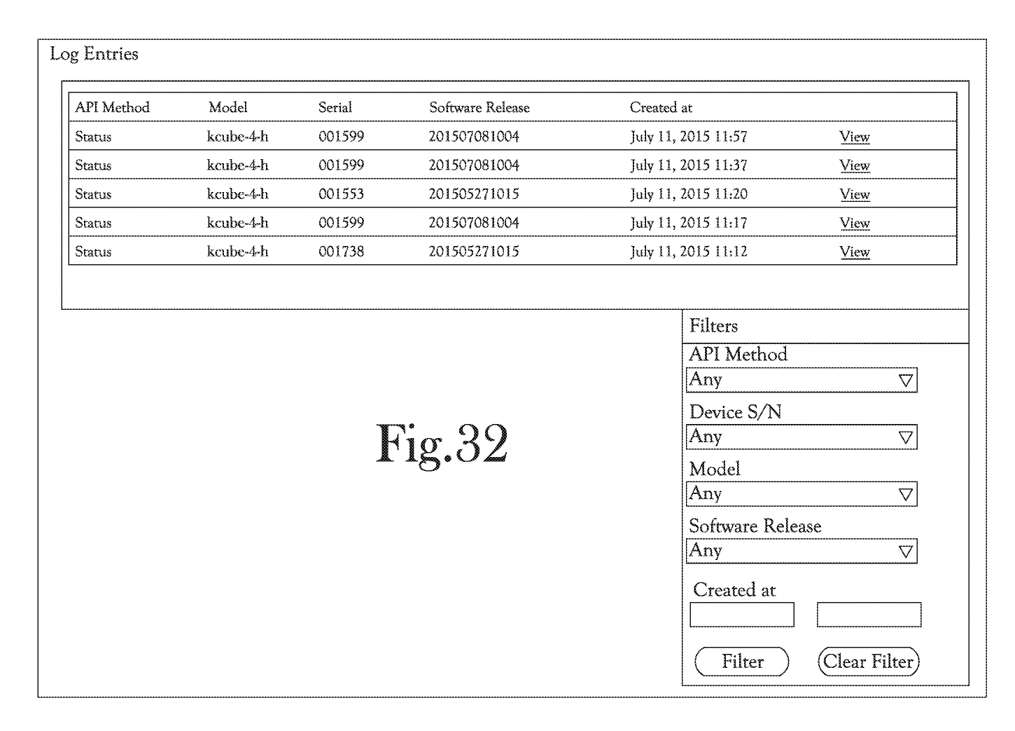

FIG. 32 illustrates a “log entries” display of an administrator interface used to show administrators laser emitting device activity according to an example embodiment of the present general inventive concept. The example embodiment of the log entries page allows administrators to see a real-time view of the activity of every laser emitting apparatus in the on demand program as they connect with the web API. In various example embodiments, a record of each data transaction may be recorded (for example, on an associated server) and archived for a certain period, e.g., six months. According to various example embodiments, a status update may be sent by the laser emitting devices according to a predetermined time cycle, e.g., every 20 minutes, to the web API. If the laser emitting device is offline, in various example embodiments the information will be updated when reconnected to the internet. In various example embodiments, the recorded data of the log entries may include the device status, purchases (which may include offers), and the on demand connection (synchronization/treatment history). Different example embodiments may record more or less data. In various example embodiments, the on demand connection data may include, for example, the treatment expiration, ID, time, and date; the device serial number, model, session ID, and status; the treatment type/patient type, size, etc.; the pain level/chronicity; and the power/time/wavelength/energy per treatment cycle. In the example log entries page illustrated in FIG. 32, the various displayed fields include API method of the laser emitting device connection, the model of the laser emitting device, the serial number, the software release under which the device is operating, and the time of creation of the current status. As with some of the similar administrator pages according to the present general inventive concept, the administrator may use a displayed filter menu to determine a range of devices to be displayed. In the example embodiment illustrated in FIG. 32, the fields which may be filtered include the API method, the device serial number, the model, the software release used by the device, and the creation time of the status.

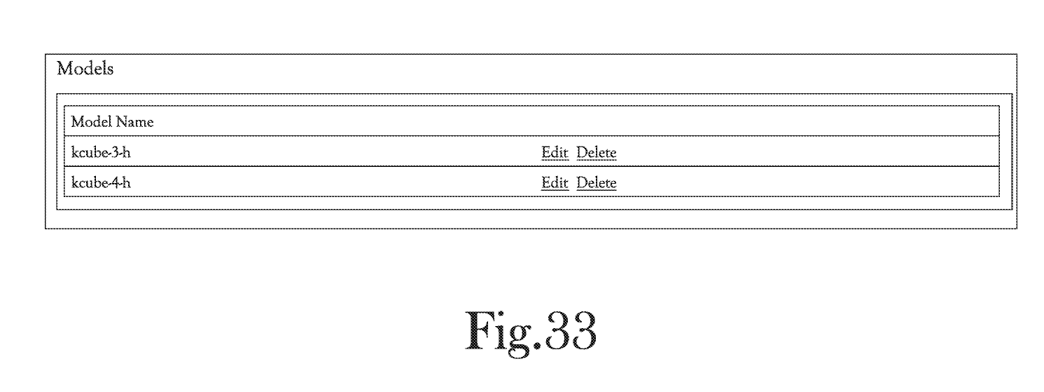

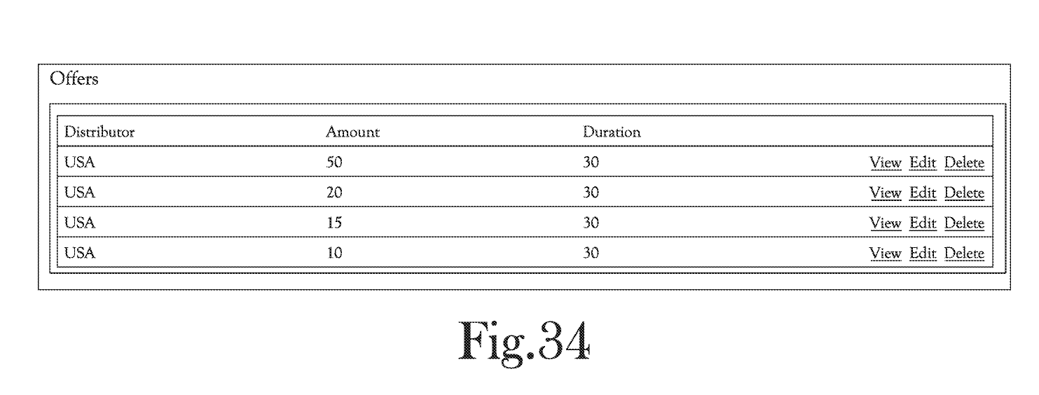

FIG. 33 illustrates a “models” display of an administrator interface used to show/enter model numbers of associated laser emitting devices according to an example embodiment of the present general inventive concept. From time to time, the hardware components of the laser emitting devices may change by various degrees to accommodate new functions. Such changes dictate a change in model number to differentiate the newly equipped device from the previous iteration. The models page of the example embodiment illustrated in FIG. 33 provides administrators the ability to enter new on demand and other converted Wi-Fi devices into the system by model number. In various example embodiments, only administrators of a certain access level, such as global and full access administrators, may enter such information using the models page. The model number indicates what update information will be served to the respective device through an update file, and helps determine what information is collected by the API during a call.

FIG. 34 illustrates an “offers” display of an administrator interface used to set increments in which treatments are offered according to an example embodiment of the present general inventive concept. In the example embodiment of an offers page illustrated in FIG. 34, global and full access administrators in the United States can set the increments in which treatments are offered on the laser emitting apparatus and on the User Dashboard of the web API. In other words, the administrator is able to set the packages or bundles of treatments that a user will see on the user's menu when purchasing treatment packages. The Administrator is able to adjust several different aspects of the offers as shown on the offers page, including the distributor, the numerical amount (which may be adjusted to match the currency type in the distributor profile), the duration for which the credits purchased are valid (which is typically shown in days, but may be in other time increments in other various example embodiments), such as for 30 days as shown in FIG. 34. Such ready access and adjustability allows an administrator to easily adjust offers to help meet marketing strategies and goals.

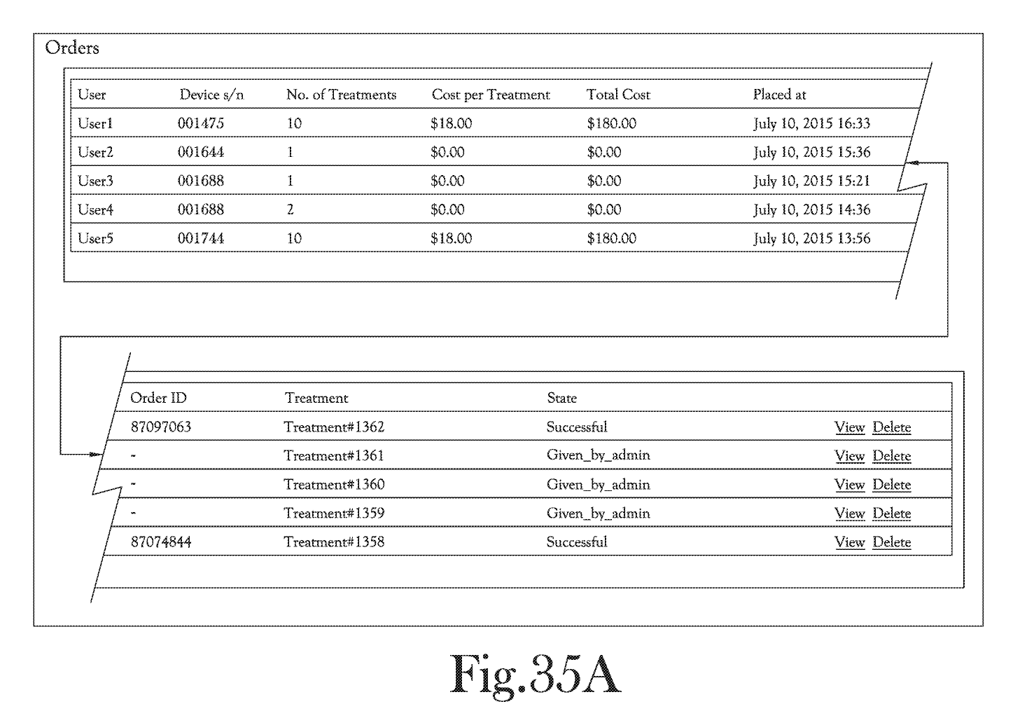

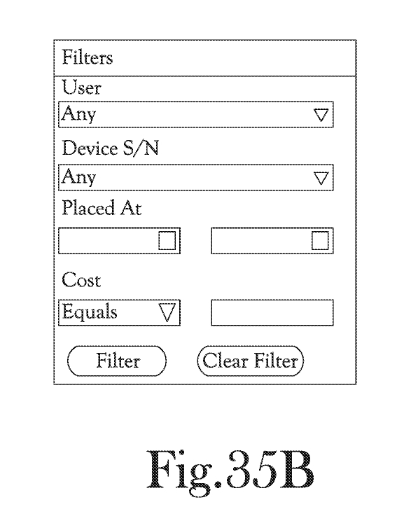

FIGS. 35A-35B illustrate an “orders” display of an administrator interface used to manage customer and administrator credited orders according to an example embodiment of the present general inventive concept. The illustrated rows of data in FIG. 35A have been split to be more easily read, and the corresponding filter screen is illustrated in FIG. 35B. In the orders page of the example embodiment illustrated in FIG. 35A, administrators with the proper level of access, such as global and full access administrators, are able to manage orders that have been credited through customers or administrators. Every credit transaction is recorded and stored in the remote device management system. Transactions may be deleted if an error occurs to maintain system reporting data integrity. As illustrated in FIGS. 35A-35B, various parameters recorded and displayed from other sections of the API include the user (username); device serial numbers; number of treatments (purchases/credited); cost per treatment (tier dependent); total cost; time and date of order; order ID (for purchase transactions); type of treatment (from treatment detail, identified by number); and state of the treatment (successful, given by administrator, etc.). FIG. 35B illustrates a filter menu that may also be included in the orders page for narrowing down the presented data. Similar to filter boxes in other screens described herein, the filter menu in FIG. 35B includes a field for choosing a range of users, a range of device serial numbers, order times and dates (“place at”), and cost. It is understood that in this orders page, as well as the other pages illustrated and described in relation to the present general inventive concept, the number and types of columns may vary according to different various example embodiments without departing from the scope of the present general inventive concept.

FIG. 36 illustrates a “page templates” display of an administrator interface used to modify user dashboards according to an example embodiment of the present general inventive concept. In the example embodiment of the page templates page illustrated in FIG. 36, different sections of the dashboard that is viewable at the user's end are selectable by the administrator for editing. In various example embodiments, administrators with the requisite access level (e.g., global and full access administrators) can modify the language in certain sections of the user dashboard, including the revenues block, footer information, and sign in page using an editor such as, for example, a WYSIWYG editor. Similar to a CMS, in various example embodiments the control over this function may be minimal, and the primary page source code may be hidden from administrator view, to ensure that the integrity of the web API is maintained.

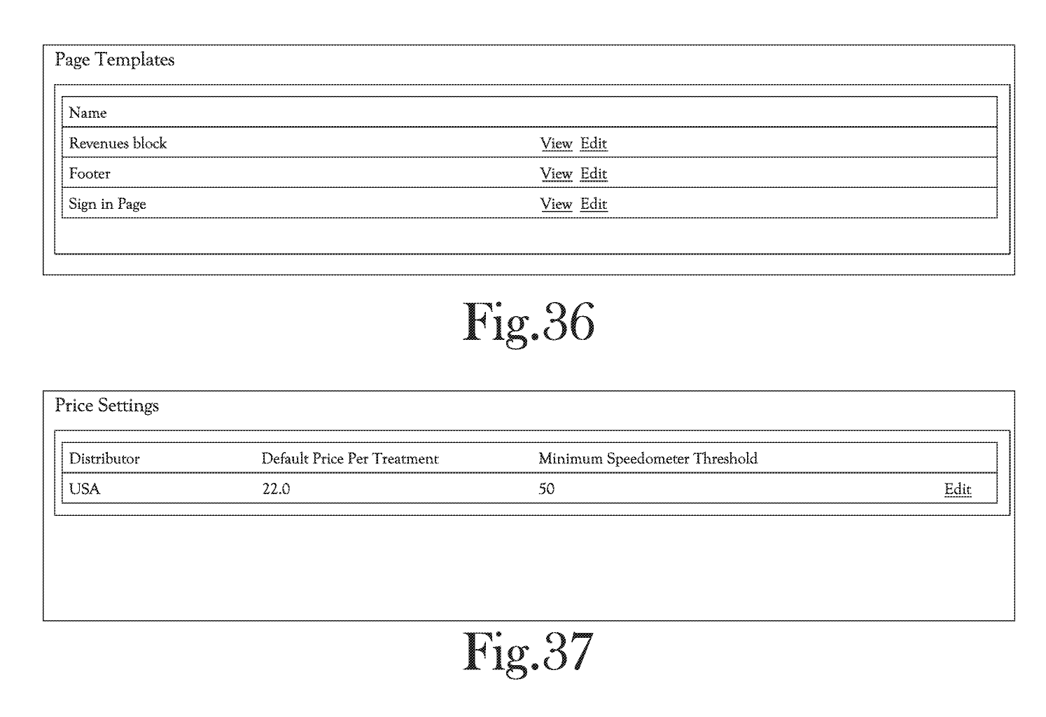

FIG. 37 illustrates a “price settings” display of an administrator interface used to adjust price settings on user dashboards according to an example embodiment of the present general inventive concept. In the example embodiment illustrated in FIG. 37, an administrator with the requisite access level (e.g., global and full access administrators) may use the interface to set the default treatment price for the system, such as by the distributor, and modify the treatment speedometer minimums displayed on the user dashboard using the price setting editing feature. It is understood that the treatment speedometer is simply one type of graphic that may be displayed on the user end to easily indicate data related to treatments used, paid for, associated prices, etc. In the example embodiment illustrated in FIG. 37, the default price per treatment for the U.S.A. distributor is 22.0 with a minimum speedometer threshold of 50. An administrator with the required level of authorization (e.g., access level) can use the edit feature to change either of the fields to increase incentives, reward users, etc.

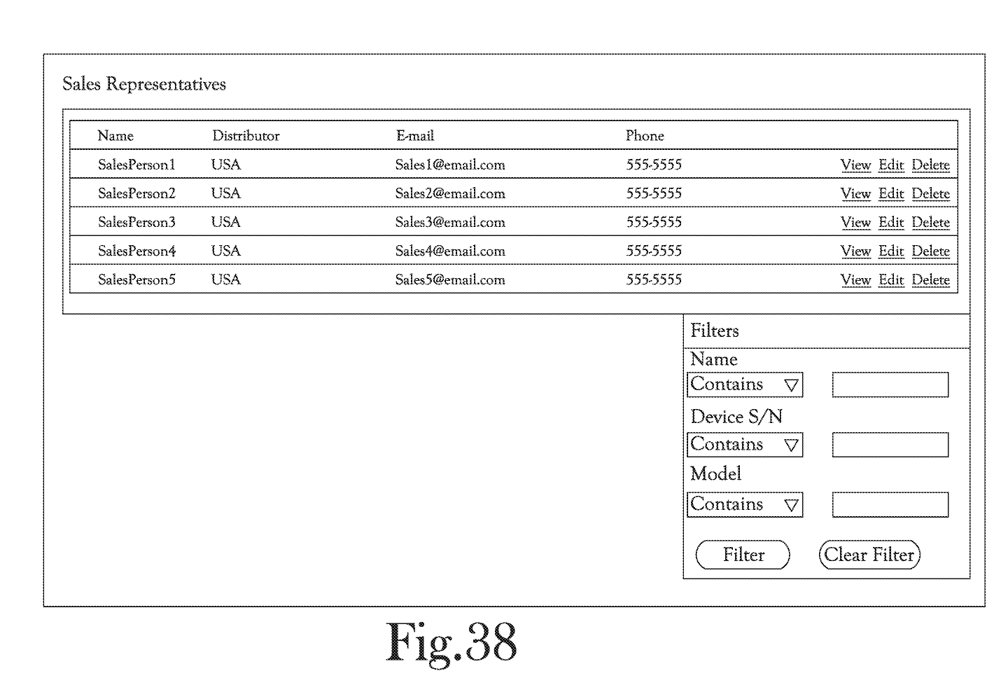

FIG. 38 illustrates a “sales representatives” display of an administrator interface used to manage sales representative data according to an example embodiment of the present general inventive concept. In the example embodiment illustrated in FIG. 38, an administrator can manage sales representatives in the system to, for example, assign responsibility for users, determine commission eligibility, report commissions once minimum user purchase thresholds are met, and the like. In the example embodiment page illustrated in FIG. 38, the name of each sales person or entity is associated with a distributor, email address, and phone number, though more or less information could be displayed in other example embodiments. Also, similar to other pages discussed herein, a filter menu is available to filter the results shown, the filter fields including the name of the salesperson, device serial numbers, model numbers, etc. In various example embodiments, sales representative information may be viewable by distributor via a menu bar on the page, such as above the “name” field. In various example embodiments, the default view may be the United States distributors, and may be accessible by administrators having the proper access level (e.g., global administrators).

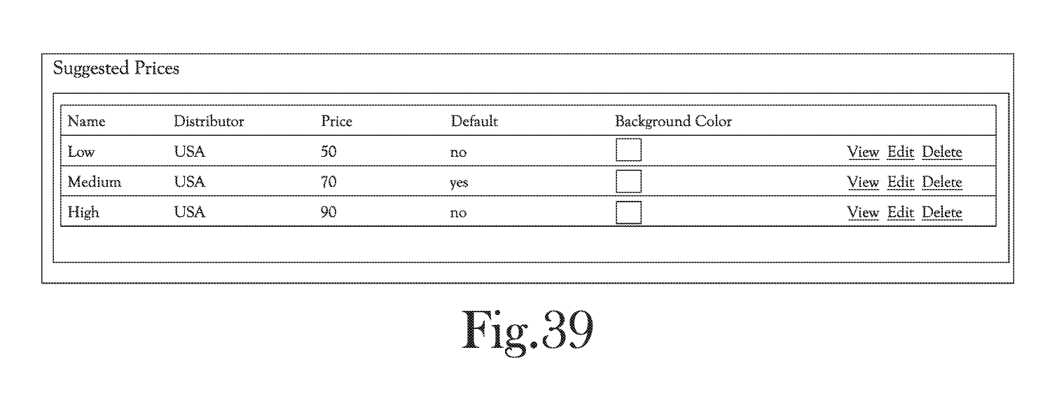

FIG. 39 illustrates a “tiers” display of an administrator interface used to set prices according to numbers of treatments purchased according to an example embodiment of the present general inventive concept. In the example embodiment illustrated in FIG. 39, tiers of suggested prices can be set for three different groupings of purchases: low; medium; and high. According to various example embodiments of the present general inventive concept, the tiers may be determined by the total number of treatments given by a user in a 30-day cycle. The displayed information, which may be set/edited by an administrator with the proper level of access (e.g., global and full access administrators), can be easily edited on the tiers page, and determines the thresholds displayed on the speedometer in the user dashboard. The tiers may be adjustable by the upper bound of quantity, color, etc., and may be viewed, edited, or deleted to change the way the speedometer is displayed. Although 3 tiers are illustrated in the example embodiment illustrated in FIG. 39, it is understood that more or fewer tiers may be set/available in other various example embodiments of the present general inventive concept.

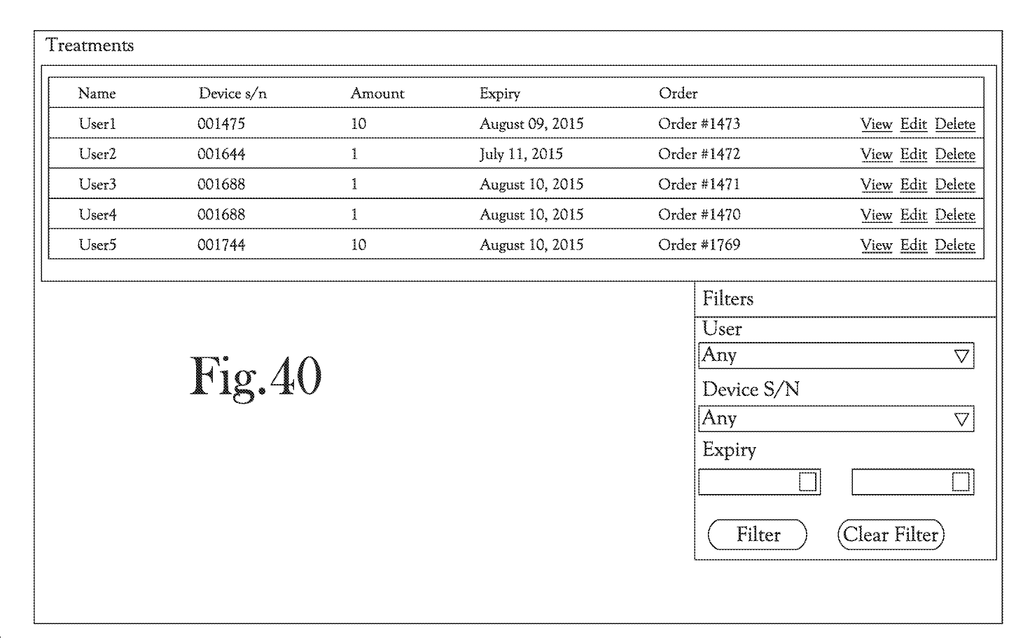

FIG. 40 illustrates a “treatments” display of an administrator interface used to edit user treatment information according to an example embodiment of the present general inventive concept. In the example embodiment illustrated in FIG. 40, the treatments page may be where administrators with the proper access level (e.g., global and full access administrators) can edit user treatments, disburse free credits, delete credits that were given or purchased in error, etc. The parameters recorded and displayed from the treatments page and other portions of the API may vary according to different example embodiments. In the illustrated embodiment of FIG. 40, a user's username is displayed along with an associated device serial number, an amount of treatments purchased and/or credited, expiration date for the credits, and order number (which may be a unique system order number). The administrator may employ the view, edit, and delete buttons to manage the data accordingly. Further, a filter menu may be displayed to provide the administrator with a way to control the users and associated information on the treatments page. For example, the filter menu illustrated in FIG. 40 allows the administrator to filter the users by various attributes, filter a range of device serial numbers, and expiration dates (marked as Expiry in FIG. 40). The treatments page allows the administrator to easily adjust the amount of treatments credited, as well as the expiration date in some example embodiments.

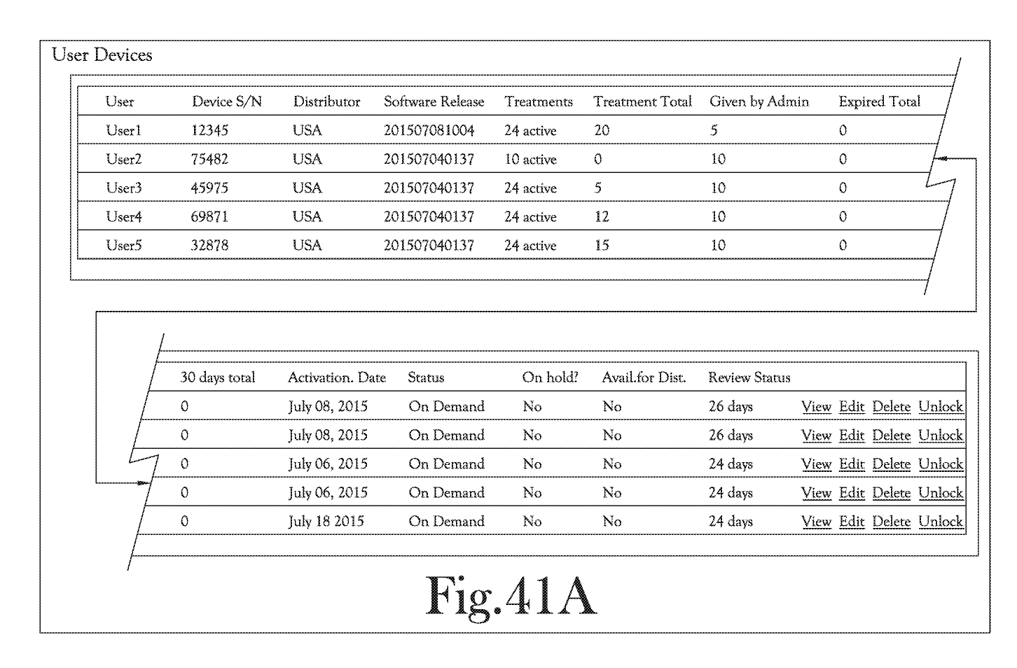

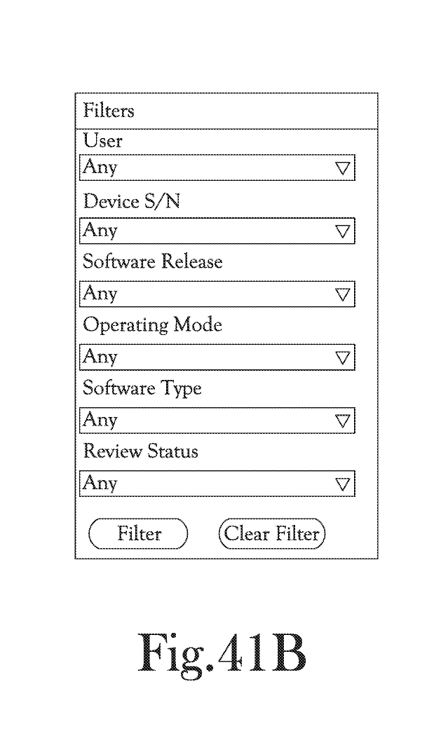

FIGS. 41A-41B illustrate a “user devices” display of an administrator interface used to display and manage portable laser emitting device (apparatus) related information according to an example embodiment of the present general inventive concept. In various example embodiments, the user devices page may be considered the heart of the web API interface, and is where the portable laser emitting apparatus is input into the system. The entered information determines user cycle dates and allows administrators with the required level of access (e.g., global and full access administrators) to monitor, adjust, and/or suspend user and device activity. For example, devices may be unlocked or made available for a distributor when a full purchase is made. According to various example embodiments, the various parameters recorded and displayed in the user devices page from other sections of the API may include a user's username, the device serial number, the distributor, the software release version number, the number of treatments authorized for the device as of the last synchronization of the device, the total number of treatments used by the user in the entire program, the type of treatments (from treatment detail), the expired total of treatments by the user in the entire program, the total number of treatments per 30-day cycle, the activation date (which may determine the start of the 30-day cycle), the status (on demand, free, unassigned, etc.), whether the device has an “on hold” status, whether the device is available for the distributor, and the review status (days remaining in the 30-day cycle). Similar to other administrator interface pages described herein, view, edit, and delete, etc., fields may be provided to the administrator for easy editing of the information. FIG. 41B illustrates a filter menu that may be included in the user devices page, by which the administrator may filter the displayed information by, for example, usernames, device serial numbers, software release versions, operating modes, software types, and review status. It is noted that while 30-day cycles are described in most of the example embodiments discussed herein, other various example embodiments may employ different time periods for the same determinations, or different time periods along with the 30-day periods.

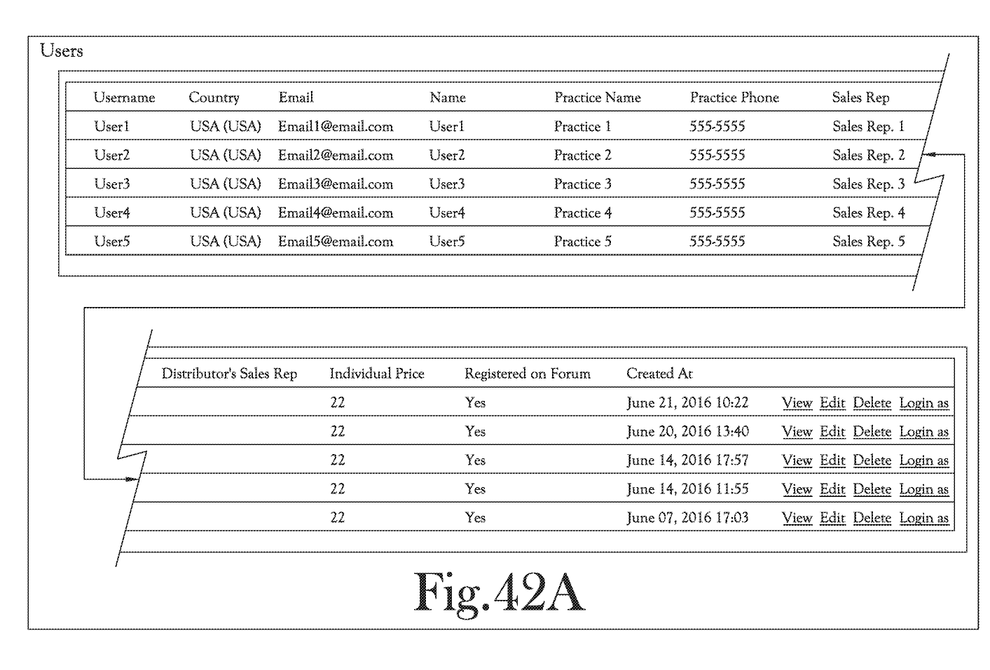

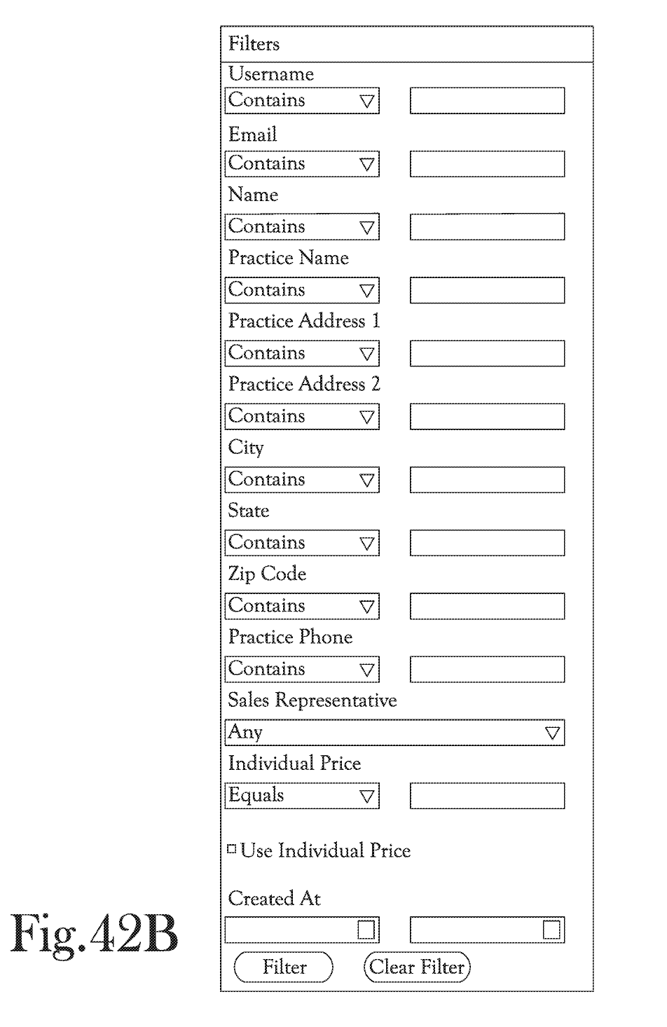

FIGS. 42A-42B illustrate a “user devices” display of an administrator interface used to display and manage live user data according to an example embodiment of the present general inventive concept. In the example embodiment page illustrated in FIGS. 42A-42B, a list of approved users may be displayed with live user data as it is stored and appears in the web API. In various example embodiments, the contact and user profile information may be managed by administrators with the required level of access (e.g., global and full access administrators), and may contain parameters that can be updated by user themselves through a user interface such as the user dashboard. Access to a user forum and pricing may be set in the edit field. Administrators with the requisite level of access may login to individual user dashboards while discussing the program with the users. As illustrated in FIGS. 42A-42B, various parameters recorded and displayed from this and other sections of the API may include the user's username, the user's country (which may determine territorial rights of distributors), the user's primary contact email address, the user's name, the practice name of the user's organization/office/etc., the phone number of the user's practice, the sales representative associated with the user, the distributor's sales representative (when applicable), the individual treatment price (which may determine two levels of amenities available to users), an indication as to whether the user is registered on the forum, the date and time at which the user profile was created, and so on. FIG. 42B illustrates an example embodiment of a filter menu that may be displayed on the users page so that the administrator can control various ranges of fields that are displayed on the page. As illustrated in FIG. 42B, the filterable fields may include, for example, usernames, email addresses, proper names of the users, names of the users' practices, address information for the practices, phone numbers for the practices, associated sales representatives, individual prices, profile creation dates, and so on.

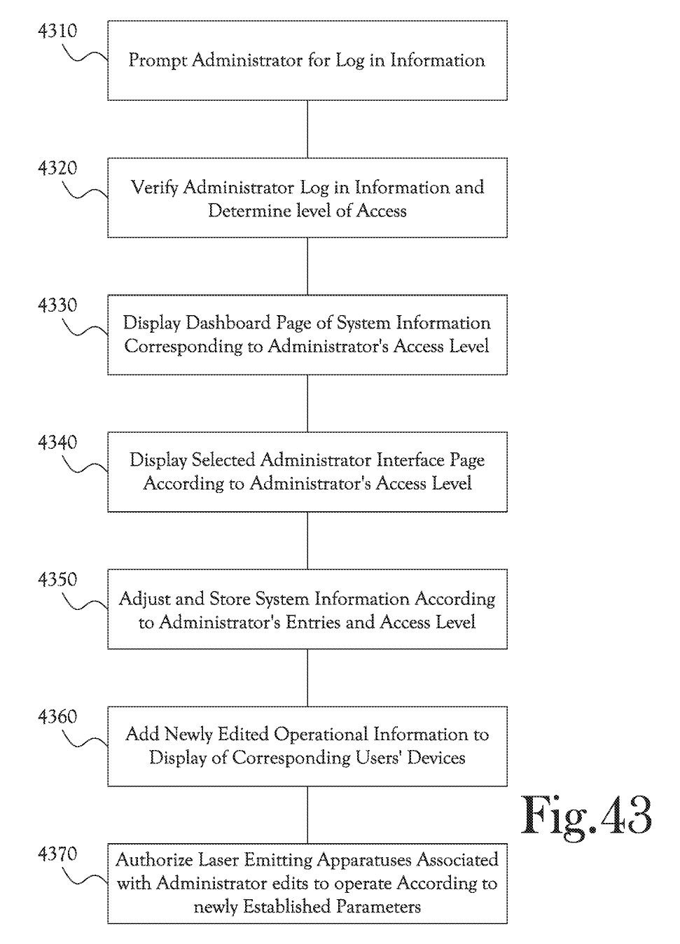

Thus, according to various example embodiments of the present general inventive concept, the portable laser emitting apparatus may be controlled according to treatment credits that may be authorized, for example, by a user buying credits through an interface on the apparatus or other communicative information processing device that is able to communicate computer data to the API of a remote computer system, and according to treatment credits that are manipulated, authorized, etc., or otherwise processed by administrators operating in the remote computer system. In an example method of an administrator adjusting purchase credit or other data associated with a user's laser emitting device, the administrator may control certain data according to his/her previously granted level of access. FIG. 43 illustrates a method of controlling the portable laser emitting apparatus according to another example embodiment of the present general inventive concept. As illustrated in FIG. 43, an administrator may be prompted for login information in operation 4310. In operation 4320, the computer system may verify the administrator login information and determine the administrator's level of access, e.g., the administrator's authorization to change data within the system, according to stored identification information corresponding to the administrator's login information. In operation 4330 the computer system may display a default administrator interface page such as a dashboard page of system information, which may also correspond to the administrator's access level. It is understood that various example embodiments of the present general inventive concept may include more or fewer operations that those illustrated in FIG. 43, and/or may be presented in different orders. The dashboard or other default administrator interface page may list various data according to users or organized classes of users, and may present a host of selectable operations that the administrator may choose from to pull up additional displays of desired information. In operation 4340 the computer system may display an administrator interface page that corresponds to a button selected by the administrator, as well as to the administrator's access level. In operation 4350, when an administrator uses one or more various editing methods to change system data that is stored in the computer system, the computer system accordingly adjusts and stores system information according to the administrator's entries and access level. In other words, if the administrator has demonstrated the requisite level of authorization (access), the fields of data edited by the administrator may be saved in one or more memories of the computer system, and the former corresponding entries may be written over with the newly edited data. In operation 4360, any applicable newly edited operational information that is to be displayed at the user device or other user interface will be added to that device. For example, if the administrator changes a quantity, price, etc., to incentivize the user to purchase more treatments, graphics indicating such information may be displayed when the user next accesses the user interface, or when the device is synchronized with the computer system, and so on. Similarly, in operation 4370 the computer system may authorize the portable laser emitting apparatuses associated with the administrator edits to operate according to the newly established parameters. For example, if the administrator has added a bonus number of treatments due to a previous error, or through some other channel of customer satisfaction, the apparatus will be authorized during the next synchronization to include the bonus treatments in the authorized number of remaining treatments. In other examples, the expiration date of one or more treatments may be extended, and so on.

Many of the operations described as being made available to administrators may be partially or fully performed automatically by the computer system managing the remote laser emitting apparatus.

According to various example embodiments of the present general inventive concept, a method of using a computer system to manage a plurality of remote laser emitting apparatuses used in medical treatments may be provided, the method including the computer system receiving, through computer data communication, apparatus identification information associated with each of a plurality of remotely located laser emitting apparatuses, and user identification information associated with a respective user of each of the laser emitting apparatuses, the computer system storing the apparatus identification information and user identification information, the computer system displaying, through an administrator graphical user interface (GUI), operational information corresponding to the plurality of laser emitting apparatuses to a system administrator, the computer system correlating, in response to receiving adjusted operational information from the system administrator through the administrator GUI, the adjusted operational information with any of the corresponding laser emitting apparatuses associated with the adjusted operational information, and the computer system communicating the adjusted operational information to the corresponding laser emitting apparatuses through computer data communication, wherein the adjusted operational information authorizes the corresponding laser emitting apparatuses to perform treatments corresponding to the adjusted operational information. The administrator GUI may be a web page displayed on an information processing device. The method may further include the computer system prompting the system administrator for system administrator identification information at an initial display of the administrator interface, and displaying operational information according to an access level associated with the entered system administrator identification information. The method may further include displaying an administrator dashboard having information regarding top and/or lowest users of the laser emitting apparatuses. The dashboard may present the information regarding top and/or lowest users of the laser emitting apparatuses as bar graphs. The may further include, in response to the entered system administrator identification information being associated with a predetermined access level, authorizing updating of certain administrative information managed by the computer system according to entries from the system administrator. The method may further include indicating administrative information that the system administrator is authorized to update with an edit marker in the administrator GUI. The method may further include displaying selectable buttons in the administrator GUI indicating other pages of administrator information that the system administrator is authorized to view. The other pages of administrator information may include an administrators page listing system administrators and associated data fields including access level, number of sign-ins, date created, or any combination thereof. The other pages of administrator information may include an email templates page having selectable email templates to be sent to users at certain expected events. The other pages of administrator information may include a firmware updates page listing software and/or firmware updates available for the laser emitting apparatuses according to model numbers of the laser emitting apparatuses. The other pages of administrator information may include a log entries page listing certain performance information of each of the laser emitting apparatuses, the performance information including model number, serial number, software release, creation date, or any combination thereof. The other pages of administrator information may include an order page listing order information from the laser emitting apparatuses, the order information including associated usernames, device serial numbers, numbers of treatments, cost per treatment, total cost, date and time of order, order ID, treatment number, treatment status, or any combination thereof. The other pages of administrator information may include a sales representatives page listing sales representatives and associated information including name, distributor, email address, phone number, or any combination thereof. The other pages of the administrator information may include a user devices page listing the laser emitting apparatuses and associated information including username, device serial number, distributor, software release, number of treatments available, total number of treatments, number of treatments given by administrators, number of expired treatments, activation date, status, or any combination thereof. The other pages of the administrator information may include a users page listing users of the laser emitting apparatuses and associated information including username, country, email address, name, practice name, practice phone, sales representative, individual treatment price, forum registration status, account creation date, or any combination thereof. The computer data communication may be communicated via the internet. Thus, the technical problem of controlling the use of a plurality of laser emitting apparatuses located far and wide across one or more regions may be solved by providing a computer system to manage the remotely located laser emitting apparatuses, to compile identification and performance information associated with the laser emitting apparatuses, and to allow administrators to access and edit various processes associated with the system and laser emitting apparatuses in real time such that remotely controlled apparatuses may be also controlled in real time.

According to various example embodiments of the present general inventive concept, a non-transitory storage medium having recorded thereon machine readable instructions to cause a computer system to perform a method of managing a plurality of remote laser emitting apparatus used in medical treatments may be provided, the method including the computer system receiving, through computer data communication, apparatus identification information associated with each of a plurality of remotely located laser emitting apparatuses, and user identification information associated with a respective user of each of the laser emitting apparatuses, the computer system storing the apparatus identification information and user identification information, the computer system displaying, through an administrator graphical user interface (GUI), operational information corresponding to the plurality of laser emitting apparatuses to a system administrator, the computer system correlating, in response to receiving adjusted operational information from the system administrator through the administrator GUI, the adjusted operational information with any of the corresponding laser emitting apparatuses associated with the adjusted operational information, and the computer system communicating the adjusted operational information to the corresponding laser emitting apparatuses through computer data communication, wherein the adjusted operational information authorizes the corresponding laser emitting apparatuses to perform treatments corresponding to the adjusted operational information.

According to various embodiments of the present general inventive concept, a portable laser emitting apparatus is provided which may be readily moved from location to location in a home or office, either in the same room or different rooms. The portable laser emitting apparatus is constructed such that it is lightweight enough to be easily moved by hand, and through a provided waveguide conveniently transmit laser light from one or more laser sources to a patient.

It is noted that the simplified diagrams and drawings do not illustrate all the various connections and assemblies of the various components, however, those skilled in the art will understand how to implement such connections and assemblies, based on the illustrated components, figures, and descriptions provided herein, using sound engineering judgment.

Various example embodiments of the present general inventive concept described herein may include operations performed by one or more processors, computers, etc., that are caused to perform these operations by instructions recorded on a non-transitory computer readable storage medium. Various ones of the operations and processes described and/or associated with the described various operations and processes may be performed on any of a host of devices, such as an online server, personal computer, smart phone, tablet computer, etc., or any device containing one or more processors to process such instructions.

Program instructions to perform a method described herein, or one or more operations thereof, may be recorded, stored, or fixed in one or more non-transitory computer-readable storage media. The program instructions may be implemented by a computer. For example, the computer may cause a processor to execute the program instructions. The media may include, alone or in combination with the program instructions, data files, data structures, and the like. Examples of computer-readable media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The program instructions, that is, software, may be distributed over network coupled computer systems so that the software is stored and executed in a distributed fashion. For example, the software and data may be stored by one or more computer readable recording mediums. Also, functional programs, codes, and code segments for accomplishing the example embodiments disclosed herein can be easily construed by programmers skilled in the art to which the embodiments pertain based on and using the flow diagrams and their corresponding descriptions as provided herein. Also, the described unit to perform an operation or a method may be hardware, software, or some combination of hardware and software. For example, the unit may be a software package running on a computer or the computer on which that software is running.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

While the present general inventive concept has been illustrated by description of several example embodiments, it is not the intention of the applicant to restrict or in any way limit the scope of the inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings.

1. A method of using a computer system to manage a plurality of remote laser emitting apparatuses used in medical treatments, comprising:
the computer system receiving, through computer data communication, apparatus identification information associated with each of a plurality of remotely located laser emitting apparatuses, and user identification information associated with a respective user of each of the laser emitting apparatuses;
the computer system storing the apparatus identification information and user identification information;
the computer system displaying, through an administrator graphical user interface (GUI), operational information corresponding to the plurality of laser emitting apparatuses to a system administrator;
the computer system correlating, in response to receiving adjusted operational information from the system administrator through the administrator GUI, the adjusted operational information with any of the corresponding laser emitting apparatuses associated with the adjusted operational information; and
the computer system communicating the adjusted operational information to the corresponding laser emitting apparatuses through computer data communication;
wherein the adjusted operational information authorizes the corresponding laser emitting apparatuses to perform treatments corresponding to the adjusted operational information.

2. The method of claim 1, wherein the administrator GUI is a web page displayed on an information processing device.

3. The method of claim 1, further comprising the computer system prompting the system administrator for system administrator identification information at an initial display of the administrator interface, and displaying operational information according to an access level associated with the entered system administrator identification information.

4. The method of claim 3, further comprising displaying an administrator dashboard having information regarding top and/or lowest users of the laser emitting apparatuses.

5. The method of claim 4, wherein the dashboard presents the information regarding top and/or lowest users of the laser emitting apparatuses as bar graphs.

6. The method of claim 3, further comprising, in response to the entered system administrator identification information being associated with a predetermined access level, authorizing updating of certain administrative information managed by the computer system according to entries from the system administrator.

7. The method of claim 6, further comprising indicating administrative information that the system administrator is authorized to update with an edit marker in the administrator GUI.

10. The method of claim 3, further comprising displaying selectable buttons in the administrator GUI indicating other pages of administrator information that the system administrator is authorized to view.

11. The method of claim 10, wherein the other pages of administrator information includes an administrators page listing system administrators and associated data fields including access level, number of sign-ins, date created, or any combination thereof.

12. The method of claim 10, wherein the other pages of administrator information includes an email templates page having selectable email templates to be sent to users at certain expected events.

13. The method of claim 10, wherein the other pages of administrator information includes a firmware updates page listing software and/or firmware updates available for the laser emitting apparatuses according to model numbers of the laser emitting apparatuses.

14. The method of claim 10, wherein the other pages of administrator information includes a log entries page listing certain performance information of each of the laser emitting apparatuses, the performance information including model number, serial number, software release, creation date, or any combination thereof.

15. The method of claim 10, wherein the other pages of administrator information includes an order page listing order information from the laser emitting apparatuses, the order information including associated usernames, device serial numbers, numbers of treatments, cost per treatment, total cost, date and time of order, order ID, treatment number, treatment status, or any combination thereof.

16. The method of claim 10, wherein the other pages of administrator information includes a sales representatives page listing sales representatives and associated information including name, distributor, email address, phone number, or any combination thereof.

17. The method of claim 10, wherein the other pages of the administrator information includes a user devices page listing the laser emitting apparatuses and associated information including username, device serial number, distributor, software release, number of treatments available, total number of treatments, number of treatments given by administrators, number of expired treatments, activation date, status, or any combination thereof.

18. The method of claim 10, wherein the other pages of the administrator information includes a users page listing users of the laser emitting apparatuses and associated information including username, country, email address, name, practice name, practice phone, sales representative, individual treatment price, forum registration status, account creation date, or any combination thereof.

19. The method of claim 1, wherein the computer data communication is communicated via the internet.

20. A non-transitory storage medium having recorded thereon machine readable instructions to cause a computer system to perform a method of managing a plurality of remote laser emitting apparatus used in medical treatments, the method comprising:
the computer system receiving, through computer data communication, apparatus identification information associated with each of a plurality of remotely located laser emitting apparatuses, and user identification information associated with a respective user of each of the laser emitting apparatuses;
the computer system storing the apparatus identification information and user identification information;
the computer system displaying, through an administrator graphical user interface (GUI), operational information corresponding to the plurality of laser emitting apparatuses to a system administrator;
the computer system correlating, in response to receiving adjusted operational information from the system administrator through the administrator GUI, the adjusted operational information with any of the corresponding laser emitting apparatuses associated with the adjusted operational information; and
the computer system communicating the adjusted operational information to the corresponding laser emitting apparatuses through computer data communication;
wherein the adjusted operational information authorizes the corresponding laser emitting apparatuses to perform treatments corresponding to the adjusted operational information.
